(19) United States
(12) Patent Application Publication
(10) Pub. No.: US 2005/0281778 A1
(43) Pub. Date: Dec. 22, 2005

(54) Human growth hormone conjugated with biocompatible polymer

(76) Inventors: Myung-Ok Park, Seoul (KR); John W. Jacobs, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/187,522

(22) Filed: Jul. 22, 2005

Related U.S. Application Data
(63) Continuation-in-part of application No. 10/947,513, filed on Sep. 22, 2004.
Application No. 10/947,513 is a continuation-in-part of application No. PCT/KR04/00701, filed on Mar. 27, 2004.

(30) Foreign Application Priority Data
Feb. 6, 2004 (KR) 10-2004-0007983
Mar. 28, 2003 (KR) 10-2003-0019734

(51) Int. Cl. A61K 38/22

(52) U.S. Cl. 424/78.27; 525/54.1

(57) ABSTRACT

The present invention relates to conjugates of biocompatible polymers and hGH, particularly PEG-hGH, where the activated biocompatible polymer is conjugated to a carboxyl group of hGH at a molar ratio of 1:1, methods of preparation, and related pharmaceutical compositions. The PEG-hGH conjugates have up to 20% of the activity of the native hGH while the in vivo half life is increased 10 fold. The PEG-hGH conjugates may be used therapeutically to treat growth retardation or growth failure, especially short stature in children, and conditions related to aging.

### CROSS-REFERENCE TO RELATED APPLICATIONS

This application is a continuation-in-part of U.S. application Ser. No. 10/947,513, filed Sep. 22, 2004 which is a continuation-in-part of International Application No. PCT/KR2004/000701, filed Mar. 27, 2004 which designates the United States and claims priority to Korean Patent Application No. 10-2004-0007983, filed Feb. 6, 2004 and Korean Patent Application No. 10-2003-0019734, filed Mar. 28, 2003.

### Reference to Sequence Listing, Table, or Computer Program Listing

A sequence listing is included as page 39.

### BACKGROUND OF THE INVENTION

1. Field of the Invention

Embodiments of the present invention relate to Human Growth Hormone (hGH) which is conjugated with a biocompatible polymer at a molar ratio of 1:1, methods of preparation thereof and pharmaceutical compositions and kits comprising the same. Therapeutic treatment methods are also disclosed. Preferred embodiments of the present invention relate to conjugates formed by specifically binding polyethylene glycol (PEG) to a carboxyl group of hGH at a molar ratio of 1:1.

2. Description of the Related Art

Human growth hormone (hGH) is a single polypeptide chain composed of 191 amino acids (Goeddel D V, et al. 1979. Nature 231:542-548; Pearlman R, et al. 1993. Stability and Characterization of Human Growth Hormone. in, Stability and Characterization of Protein and Peptide Drugs: Case Histories, edited by Y J Wang and R Pearlman. Plenum Press, New York). Endogenous growth hormone is responsible for stimulating normal skeletal, connective tissue, muscle, and organ growth in children and adolescents. It also plays an important role in adult metabolism. Somatropin (recombinant human Growth Hormone) binds to growth hormone (hGH) receptors and produces a variety of physiologic effects that promote growth. Many of the biological actions of growth hormone are mediated by insulin-like growth factor-1 acting directly on the responsive tissue (Clark R. 1997. Endocrine Reviews 18:157-179).

The primary structure of human growth hormone is shown in FIG. 25 below. Endogenous hGH is produced in the anterior pituitary gland. Human growth hormone was first isolated in 1956 and its structure was identified in 1972 (Pearlman R, et al. 1993. Stability and Characterization of Human Growth Hormone. in, Stability and Characterization of Protein and Peptide Drugs: Case Histories, edited by Y J Wang and R Pearlman. Plenum Press, New York). Prior to 1985, growth hormone was derived from human cadavers, but the cloning and expression of human growth hormone in the late 1970's led to the availability of several marketed hGH products that mimic all of the normal functions of endogenous hGH (Drake W M, et al. 2001. Endocrine Reviews 22:425-450). Genetech's Protropin® was originally approved by the FDA in the mid 1980s for treating growth failure due to growth hormone deficiency. Since then other indications for the use of hGH have been approved including growth deficiency seen in chronic renal disease, or Turner's syndrome and for treating cachexia and AIDS wasting (Drake W M, et al. ibid.).

An important advance in growth hormone therapy would be the availability of a long-acting hGH product. Currently hGH must be injected six times a week in children and a once a week injection would have a huge market impact. Genentech has marketed a long-acting formulation for hGH (Nutropin-Depot) but withdrew it from the market due to poor sales. This was due to a widespread belief among pediatric endocrinologists that the depot form of hGH was not as effective in accelerating growth rate in children. Embodiments of the present invention are directed to a more attractive approach for a long-acting growth hormone would be to pegylate the protein.

Conjugates of proteins or pharmaceutically active molecules such as hGH to biocompatible polymers can afford great advantages when they are applied in vivo and in vitro. When being covalently bonded to biocompatible polymers, biologically active materials can exhibit modified surface properties and solubility, and thus can be increased in solubility within water or organic solvents. Further, the presence of biocompatible polymers can make the proteins and/or polypeptides conjugated to them more stable in vivo, increase biocompatibility of the proteins and reduce immune response, and reduce the clearance rate of the proteins by the intestine, the kidney, the spleen, or the liver.

Although the conjugation of the biologically active materials such as a hGH with biocompatible polymers such as PEG has many advantages, problems remain in conjugating by known methods.

The most common conjugation method is achieved by bonding activated PEG to the amino group of amino acid residues such as lysine. However, because one or more free lysine residues in many proteins are frequently located at or adjacent to the active site, when a lysine residue is used for the conjugation, it tends to decrease the biological activity of the PEG-protein conjugates substantially. Furthermore, because lysine reacts easily with PEG, PEG-protein conjugates with two or more PEG molecules attached to one protein molecule are generally obtained. For example, when more than two PEG molecules bind to the surface of cytokines such as interferon, CSF, and interleukin or polypeptides such as EGF, hGH, and insulin, the biological activity of conjugate is rapidly reduced resulting in loss of function. Additionally, since these reactions tend to occur randomly, a mixture of many kinds of PEG-protein conjugates is produced, which makes the purification of the desired conjugates complicated and difficult. If too many polymer molecules are attached to targeting proteins or peptides, the conjugates lose all or much of their biological activity. Also, if an expressively reactive linker has been used or insufficient numbers of polymers are attached to targeting protein molecules, the therapeutic efficacy of those conjugates can be decreased.

To overcome these problems, attempts have been made to conjugate biocompatible polymers to amino acid residues of proteins substituted by genetic engineering to conjugate polymers to a specific site of the protein. However, this method generally alters the original properties of proteins. Also the safety of these genetically engineered molecules as therapeutic drugs needs to be determined.

Attempts have been made to solve the problems by chemically modifying specific sites of biologically active materials with biocompatible polymers. U.S. Pat. Nos. 5,951,974 and 5,985,263 describe conjugation of PEG molecules to the histidine residue of interferon to increase the efficacy of drugs by lengthening the half-life in vivo and the like. However, this method again used the reactive amino group and produced isomers of PEG-IFN randomly attached at several histidine sites, and required an additional purification step using an ion-exchange column to separate the desired 1:1 complex of highly active PEG-IFN conjugate. Further, the imidazolyl group of histidine to which PEG is attached is easily hydrolyzed compared to other amino groups of amino acids, and interferon is easily released from the PEG-interferon conjugate.

U.S. Pat. No. 5,766,897 describes conjugation of macromolecules and mutant forms thereof at their cysteine residues to activated PEG molecules. Because of disulfide bond formation, most protein molecules have either one free or no spare cysteine. An amino acid which is not related to the active site must be substituted for a cysteine residue by mutagenesis to provide a new cysteine residue for conjugation with polymers. This method, however, tends to produce conjugates with significantly decreased activity compared to conjugates at amino or carboxyl groups of proteins, although it has an advantage of attaching the polymer to a specific site on a biologically active molecule.

U.S. Pat. No. 5,985,265 describes site-specific conjugates at N-terminal residues of G-CSF and IFN with PEG molecules. However, reactivity of these activated polymers is low, and the reaction needs a longer reaction time. In addition, the yield of the reaction is low and stability of proteins is poor. When the active site of protein molecules is especially near the N-terminus, conjugation at the N-terminal amino group results in the significant decrease or loss of biological activity.

U.S. Pat. No. 5,824,778 describes conjugates of G-CSF at amino and carboxyl groups by PEG. Excess EDAC was added to activate the carboxyl groups of the protein and many PEG molecules were attached to activated carboxyl groups of several residues. The obtained PEG-G-CSF conjugate has been determined to be a heterogeneous mixture having various numbers of PEG molecules attached. The biological activity of the conjugate was significantly reduced.

Previous attempts to pegylate hGH by Genentech resulted in hGH preparations that were only 1/400 as active as the native hGH protein. This precluded the clinical development of those earlier peg-growth hormones. If the biological activity of biologically active molecules such as hGH can be maintained after conjugation with the polymer at a desired ratio, and a homogenous species of site-specific conjugates can be obtained, clinical usefulness of molecules such as hGH will increase remarkably.

### SUMMARY OF THE INVENTION

Embodiments of the invention are directed to a conjugate of biocompatible polymer-human Growth Hormone (hGH), wherein the biocompatible polymer is conjugated to a carboxyl group of hGH at a molar ratio of 1:1. In preferred embodiments, the biocompatible polymer is polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacrylamide, or copolymers thereof. More preferably, the biocompatible polymer is PEG-20000 or PEG-30000.

Embodiments of the invention are directed to pharmaceutical compositions which include a pharmaceutically acceptable amount of a conjugate of biocompatible polymer-human Growth Hormone (hGH), wherein the biocompatible polymer is conjugated to a carboxyl group of hGH at a molar ratio of 1:1 and a pharmaceutically acceptable carrier. In preferred embodiments, the biocompatible polymer is polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacrylamide, or copolymers thereof. More preferably, the biocompatible polymer is PEG-20000 or PEG-30000.

In preferred embodiments, the carboxyl group for conjugation is the C-terminus of hGH. Preferably, the activity of the conjugate is 10-20% of the activity of an unconjugated hGH protein.

Embodiments of the invention are directed to methods of preparing a conjugate of biocompatible polymer-hGH which include one or more of the following steps:

(a) activating a biocompatible polymer with the stepwise addition of a coupling reagent; and

(b) conjugating the activated biocompatible polymer to a carboxyl group of the hGH at a molar ratio of 1:1,

wherein the molar ratio of the hGH to the activated biocompatible polymer is 1:1 to 1:20, the ratio of the hGH to the coupling reagent is 1:1 to 1:50, and pH is in the range of 2 to 5.

In preferred embodiments, the biocompatible polymer is activated with a reactive functional group which is able to react with a carboxylic acid and/or a reactive carbonyl group. Preferably, the biocompatible polymer is polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacrylamide, or copolymers thereof. More preferably, the biocompatible polymer is PEG-20000 or PEG-30000. In preferred embodiments, the carboxyl group is the C-terminus of hGH.

In some embodiments, methods of preparing a conjugate of biocompatible polymer-hGH include one or more of the following additional steps:

(c) purifying the conjugate by size exclusion chromatography;

(d) precipitating the conjugate of (c) using ammonium sulfate;

(e) purifying the conjugate of (d) by ion exchanges chromatography; and

(f) purifying the conjugate of (e) by hydrophobic interaction chromatography.

In preferred embodiments, the biocompatible polymer is conjugated to a carboxyl group of the hGH at a molar ratio of 1:1. Preferably, the biocompatible polymer is PEG. Preferably, the conjugated hGH has 10-20% of the activity of unconjugated hGH protein.

Embodiments of the invention are directed to methods of treating growth failure or growth retardation by administering an effective amount of a composition which includes PEG-hGH to a patient in need thereof, wherein the PEG is conjugated to a carboxyl group of hGH at a molar ratio of 1:1. Preferably, the carboxyl group is the C-terminus of hGH. In preferred embodiments, the PEG-hGH has 10-20% of the activity of unconjugated hGH protein. Preferably, the growth failure or growth retardation is due to hormone deficiency, chronic renal disease, Turner's syndrome, cachexia or AIDS wasting. Preferably, the PEG-hGH is administered in combination with a pharmaceutically acceptable carrier. In preferred embodiments, the administration is done by injection. In alternate preferred embodiments, the administration is oral. Preferably, the composition which includes PEG-hGH is administered no more than twice per week to the patient in need thereof.

Embodiments of the invention are directed to a method of treating short stature in children by administering an effective amount of a composition which includes PEG-hGH to a patient in need thereof at a frequency of no more than twice/week, wherein the PEG is conjugated to a C-terminus carboxyl group of hGH at a molar raio of 1:1 and the PEG-hGH has 10-20% of the activity of unconjugated hGH.

Embodiments of the invention are directed to methods of treating adverse effects associated with aging including decrease in lean muscle, increase in blood pressure, increase in cholesterol, increase in body fat, loss of skin tone, and decrease in bone density by administering an effective amount of a composition which includes PEG-hGH to a patient in need thereof, wherein the PEG is conjugated to a carboxyl group of hGH at a molar raio of 1:1.

Embodiments of the invention are directed to kits which include a conjugate of biocompatible polymer-human Growth Hormone (hGH), wherein the biocompatible polymer is conjugated to a carboxyl group of hGH at a molar ratio of 1:1 and is in lyophillized form; a pharmaceutically acceptable carrier for reconstitution of the conjugate; and a delivery device for delivery of the reconstituted conjugate to a patient in need thereof.

In some embodiments, the kit may also include a skin antiseptic; and/or an instruction sheet.

Embodiments of the invention are directed to a conjugate of biocompatible polymer-human Growth Hormone (hGH), wherein the biocompatible polymer is conjugated to a carboxyl group of hGH at a molar ratio of 1:1 and preloaded in a syringe for delivery to a patient.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1 shows the degree of conjugation for mPEG(5K)-Hz-G-CSF by HPLC (FIG. 1A) and SDS-PAGE (FIG. 1B).

FIG. 2 shows the degree of conjugation for mPEG(20K)-Hz-G-CSF by HPLC (FIG. 2A) and SDS-PAGE (FIG. 2B).

FIG. 3 shows mPEG(5K)-Hz-IFN with the molar ratio of 1:1 on SDS-PAGE.

FIG. 4 shows the productivity of mPEG(20K)-Hz-IFN conjugate according to the amount of EDAC added on SDS-PAGE.

FIG. 5 shows the degree of reactivity for mPEG(20K)-Hz-IFN conjugate according to the addition method of EDAC and the amount of EDAC on SDS-PAGE.

FIG. 6 shows SDS-PAGE of mPEG(20K)-Hz-IFN conjugate purified by an ion-exchange column.

FIG. 7 shows the comparison of the biological activity of mPEG(20K)-Hz-G-CSF conjugate, native G-CSF, and Neulasta™ (PEG-G-CSF, developed by Amgen, FDA approved in 2002) by cell based assay.

FIG. 8 shows the plasma half-life of mPEG(20K)-Hz-G-CSF, native G-CSF, and Neulasta™(PEG-G-CSF, developed by Amgen, FDA approved in 2002).

FIG. 9 shows WBC of mPEG(20K)-Hz-G-CSF conjugates, native G-CSF, and Neulasta™(PEG-G-CSF, Developed by Amgen, FDA approved in 2002).

FIG. 10 shows biological activity of mPEG(12K)-Hz-IFN conjugate, native IFN, and PEG-IFN(developed by Schering-Plough) by CPE assay.

FIG. 11 shows the comparison of biological activity of mPEG(20K)-Hz-IFN conjugate with native IFN by CPE assay.

FIG. 12 shows the comparison of biological activity between Di-mPEG-Hz-IFN, two PEG molecules attached to one IFN molecule and mono-mPEG-Hz-IFN, one PEG molecule attached to one IFN molecule, by CPE assay.

FIG. 13 shows the comparison of half-life in plasma of PEG(20K)-Hz-IFN conjugate, native IFN, and PEG-IFN(developed by Schering-Plough).

FIG. 14 shows the comparison of stability between PEG-IFN conjugated at a carboxyl group (FIG. 14B) and at an amino group (FIG. 14A).

FIG. 15 shows the HPLC chromatogram for native PTH, which has not been modified by biocompatible polymers.

FIG. 16 shows the HPLC chromatogram for a reaction mixture (unreacted PTH, mPEG(20K)-Hz-PTH, mPEG(20K)-Hz) of PTH with mPEG(20k)-Hz before purification (peak 1: unreacted PTH with PEG polymer, peak 2: mPEG(20k)-Hz-PTH)

FIG. 17 shows HPLC chromatogram of the purified mPEG(20kO)-Hz-PTH after conjugating PTH with mPEG(20K)-Hz.

FIG. 18 shows SDS-PAGE, stained with Coomassie blue for the reaction product between PTH and mPEG(20k)-Hz (lane 1: MW marker, lane 2: PTH, lane 3: PTH, PTH-mPEG(20k)-Hz conjugate before purification, lane 4: PTH-mPEG(20k)-Hz conjugate after purification.

FIG. 19 shows the in vivo biological activity for PTH and PEG-PTH conjugate.

FIG. 20 shows the half-life of PTH and PEG-PTH conjugates in rats.

FIG. 21 shows the HPLC chromatogram for purified PEG-EPO with 1:1 complex.

FIG. 22 shows a cell proliferation assay of native EPO and PEG-EPO.

FIG. 23 shows SDS-PAGE gel of SYNTROPIN drug substance through successive purification steps. Lane 1: MW marker; Lane 2: hGH conditioned medium; Lane 3: (NH₄)₂SO₄ precipitated hGH medium; Lane 4: hGH purified by Q-Sepharose column; Lane 5: hGH purified by Q-Sepharose and Phenyl-Sepharose columns.

FIG. 24 shows a densitometer scan of SDS PAGE gel of purified SYNTROPIN drug substance. 99.9% of the material is in peak # 2.

FIG. 25 shows the primary structure of human Growth Hormone (hGH).

FIG. 26 shows an HPLC profile of PEGylation of hGH on a size exclusion column.

FIG. 27 shows HPLC profiles of purified mono- and di-PET-hGH on a size-exclusion column. FIG. 27A shows profile for mono-PEG-hGH. FIG. 27B shows profile for di-PEG-hGH.

FIG. 28 shows the biological activity of PEG-hGH by cell proliferation assay.

FIG. 29 shows PK study of PEG-hGH in rats (dose=200 μg/kg, s.c. injection).

FIG. 30 shows bioassay of hGH in cells expressing full-length hGH receptors.

FIG. 31 shows an animal study of hGH and peg-hGH injected into hypophysectomized rats with weight gain of the animals monitored over a 28 day period.

### DETAILED DESCRIPTION OF THE PREFERRED EMBODIMENT

Embodiments of the invention are directed to the conjugates of hGH with a biocompatible polymer, particularly PEG, where the activated biocompatible polymer is conjugated to a carboxyl group of biologically active hGH at a molar ratio of 1:1.

Embodiments of the invention are directed to a pharmaceutical composition comprising a therapeutically effective amount of the above biocompatible polymer-biologically active hGH conjugates and pharmaceutically acceptable carriers.

Embodiments of the invention are directed to a method of preparation of conjugates of biocompatible polymer-biologically active hGH at a molar ratio of 1:1, wherein the biocompatible polymer is conjugated at a carboxyl group of biologically active hGH, comprising the step of conjugating the biologically active hGH to the activated biocompatible polymer with the stepwise addition of coupling reagent under conditions where the molar ratio of biologically active hGH to activated biocompatible polymer is 1:1 to 1:20, the ratio of biologically active hGH to the coupling reagent is 1:1 to 1:50, and pH is in the range of 2 to 5.

In the above method, EDAC, as an example of the coupling reagent, was added stepwise more than 5 times, preferably 5 or 6 times, because EDAC is readily hydrolyzed in aqueous solution.

The method described above provides the conjugates wherein biocompatible polymers are attached to a carboxyl group of biologically active molecules at a ratio of 1:1. In other words, the present invention provides site specific conjugation by binding activated polymers to a carboxyl group of biologically active hGHs at a molar ratio of 1:1. These conjugates retain the biological activity of biologically active hGHs by preventing the attachment of polymers to active sites. The present invention also provides the conjugates at a molar ratio of 1:1 by avoiding the random reaction with many reactive residues at active sites to produce various kinds of heterogeneous mixtures. Further, the conjugates of the present invention have several advantages such as increased stability in vivo, increase of bioavailability, and extended half-life caused by biocompatible polymers. Therefore, production of homogeneous biocompatible polymers-biologically active hGH conjugates of the present invention provides a cost and time effective process, as compared to other processes of the prior art.

WO92/16555 describes the reaction of ovalbumin at the carboxyl or carbohydrate group with PEG-hydrazide (Hz) containing an amino acid spacer. However, it only describes the conjugates with a number of PEG molecules attached. There is no disclosure of a method of preparation for conjugates of biologically active materials such as hGHs with biocompatible polymers with the ratio of 1:1, and the biological activity of conjugates.

Also, U.S. Pat. No. 5,824,779 describes linkage of PEG to the carboxyl group of G-CSF but the conjugate prepared according to their method had very low activity because several PEG molecules were randomly attached to aspartic acids or glutamic acids of G-CSF.

There have been difficulties in providing controlled reaction conditions for specific linkages or to control the number of attaching polymers using the difference in reactivity according to the pKa of amino acids.

When an attempt was made to react the amino group of lysine in the range of pH 7 to 8, the histidine groups also reacted randomly. And, when pH was lowered to about 6 to 6.5, histidine groups became more reactive with PEG than lysine groups(U.S. Pat. No. 5,951,974 and U.S. Pat. No. 5,985,263). This problem has been solved according to embodiments of the present invention, by attachment of PEG to carboxyl groups, especially the C-terminus of biologically active materials such as hGHs at a ratio of 1:1 at a pH equal to or lower than 3.

Therefore, embodiments of the present invention relate to the conjugate of biocompatible polymer-biologically active hGH, wherein the biocompatible polymer is conjugated to the C-terminus of the biologically active hGH at a molar ratio of 1:1.

In another aspect, embodiments of the present invention relate to a pharmaceutical composition comprising a pharmaceutically acceptable amount of the conjugate, wherein the biocompatible polymer is conjugated to the C-terminus of the biologically active hGH at a molar ratio of 1:1 and pharmaceutically acceptable carriers.

Embodiments of the invention are directed to a method of preparation of a conjugate of biocompatible polymer-biologically active hGH at the C-terminus of the biologically active hGH with a molar ratio of 1:1, comprising the step of conjugating the biologically active hGH to the activated biocompatible polymer with the stepwise addition of coupling reagent under conditions where the molar ratio of biologically active hGH to the activated biocompatible polymer is 1:1 to 1:20, the ratio of biologically active hGH to the coupling reagent is 1:1 to 1:50, and pH is in the range of 2 to 5.

Biocompatible Polymers

The term “conjugating material” used for conjugation of biologically active molecules means any biocompatible polymer which can be linked to biologically active molecules such as natural or synthetic polymers.

The term “biocompatibility” means biocompatible with living tissues or systems, and being nontoxic, noninflammatory, and noncarcinogenic without causing harm, inflammation, immune response and/or carcinogenesis in the body.

Biocompatible polymers are conjugated with biologically active materials such as hGH. The useful polymers of the present invention are readily soluble in various solvents and have molecular weight of between about 300 and about 100,000 Da and preferably between about 2,000 and about 40,000 Da. The biocompatible polymers include, but are not limited to, polyethylene glycol (PEG), polypropylene glycol (PPG), polyoxyethylene (POE), polytrimethylene glycol, polylactic acid and its derivatives, polyacrylic acid and its derivatives, polyamino acid, polyvinylalcohol, polyurethane, polyphosphazene, poly(L-lysine), polyalkylene oxide (PAO), polysaccharide, dextran, polyvinyl pyrrolidone, polyacrylamide, copolymers thereof and other nonimmunogenic polymers.

Biocompatible polymers of the present invention are intended to include not only linear polymers but also polymers as follows. Biocompatible polymers of the present invention include soluble, non-antigenic polymers linked to an activated functional group that is capable of being nucleophilically substituted through an aliphatic linker residue (U.S. Pat. Nos. 5,643,575 and 5,919,455). Also, biocompatible polymers of the present invention include multi-armed, mono-functional and hydrolytically stable polymers, having two linker fragments which have polymer arms around a central carbon atom, a residue which is capable of being activated for attachment to biologically active materials such as proteins, and side chains which can be hydrogen or methyl group, or other linker fragment (U.S. Pat. No. 5,932,462). In addition, biocompatible polymers of the present invention include polymers of branched PEG in which the functional groups of polymers are attached to biologically active materials via linker arms having reporter residues (WO 00/33881).

Among them, PEG is one of the most common biocompatible polymers of the present invention. In general, PEG is a nontoxic hydrophilic polymer having the repeating unit, HO—(CH₂CH₂O)ₙ—H. Various proteins are reported to show extended half-lives, increased solubility, increased stability, and reduced immunogenicity in plasma when being conjugated with PEG.

The range of molecular weight of PEG molecules conjugated to biologically active materials such as proteins or peptides is from about 1,000 to 100,000 Da and the toxicity of PEG over 1,000 Da is known to be very low. PEGs in the range of from 1,000 to 6,000 Da are distributed to the whole body and cleared in the kidney. Branched PEG with molecular weight of 40,000 Da are distributed in blood or organs including the liver, and metabolized in the liver.

PEG is a most preferable biocompatible polymer because PEG is commercially available in the various molecular weight ranges, each oxyethylene unit is hydrophilic to be accessible to bind 2-3 water molecules, PEG derivatives with one-terminal functional group from methoxy polyethylene glycol are easy to synthesize, PEG has very low risk of antigen-antibody reaction, and the related technology is well developed.

Biologically Active Materials

The term “biologically active molecule” or “biologically active material” means all nucleophiles conjugated with activated biocompatible polymers, and which retain at least some of their biological activity after conjugation. Preferred embodiments are directed to biologically active molecules which include hGH. The term “biologically activity” used herein is not limited by physiological or pharmacological activity. For example, some conjugates of nucleophilic containing enzymes can catalyze reactions in organic solvents. Similarly, some polymer conjugates including proteins such as Con-canavalin A, or immunoglobulin can also be used in diagnostics in the laboratory. In general, biologically active molecules such as hGH can be isolated from nature or synthesized recombinantly or chemically, and include proteins, peptides, polypeptides, enzymes, biomedicines, genes, plasmids, or organic residues.

Proteins, peptides, and polypeptides of interest include, but are not limited to, hemoglobin, serum proteins (for example, blood factors including Factor VII, VIII, and IX), immunoglobulins, cytokines (for example, interleukins), α-, β- and γ-interferons, colony stimulating factors including G-CSF and GM-CSF, platelet derived growth factor (PDGF), phospholipase-activating protein (PLAP), and parathyroid hormone (PTH). Other proteins of general biological or therapeutic interest include insulin, plant proteins (for example, lectins and ricins), tumor necrosis factors (TNF) and related alleles, growth factors (for example, tissue growth factors such as TGFα and TGFβ and epidermal growth factors), hormones (for example, human growth hormone (hGH), follicle-stimulating hormone, thyroid-stimulating hormone, antidiuretic hormones, pigmentary hormones, luteal hormone-releasing hormone and derivatives thereof), calcitonin, calcitonin gene related peptide (CGRP), synthetic enkephalin, somatomedins, erythropoietin, hypothalamic releasing factors, prolactin, chorionic gonadotropin, tissue plasminogen activator, growth hormone releasing peptide (GHRP), thymic humoral factor (THF) and the like. Immunoglobulins of interest include IgG, IgE, IgM, IgA, IgD, and fragments thereof. Preferred embodiments are directed to a human Growth Hormone protein.

When two or more biocompatible polymers are attached to especially low molecular weight polypeptides such as Interferon and G-CSF, these conjugates exhibit considerably low biological activity. Also, two or more polymers are attached when relatively highly reactive amino groups are conjugated and thus the separation of conjugates in a 1:1 complex is not easy. However, in preferred embodiments the present invention provides the selective preparation of conjugates of biocompatible polymer-IFN or biocompatible polymer-G-CSF or biocompatible polymer-hGH in a 1:1 complex, wherein these conjugates show high biological activity, increased half-life, and excellent bioavailability.

The biologically active hGH of the present invention also include any portion of a polypeptide demonstrating in vivo bioactivity. This includes amino acid sequences, antisense oligomers, antibody fragments, linear antigen (Ref. U.S. Pat. No. 4,946,778), binding molecules including fusions of antibodies or fragments, polyclonal antibodies, monoclonal antibodies, catalytic antibodies, nucleotides, oligonucleotides and the like.

The biologically active materials also include enzymes. Enzymes of interest include carbohydrate-specific enzymes, proteolytic enzymes, oxidoreductases, transferases, hydrolases, lyases, isomerases and ligases. Without being limited to particular enzymes, examples of enzymes of interest include asparaginase, arginase, arginine deaminase, adenosine deaminase, superoxide dismutase, endotoxinases, catalases, chymotrypsin, lipases, uricases, adenosine diphosphatase, tyrosinases and bilirubin oxidase. Carbohydrate-specific enzymes of interest include glucose oxidases, glucosidases, galactosidases, glucocerebrosidases, glucouronidases, etc.

Examples described above are examples of suitable biologically active nucleophiles conjugated with activated biocompatible polymers of the present invention. All suitable biologically active hGHs with nucleophilic group are to be also included in the present invention although they are not mentioned above. The biologically active hGHs for the present invention need to possess at least one free carboxyl group for conjugation by polymer.

The conjugates of the present invention are biologically active for the purpose of therapeutic application. Mammals can be treated by administering the therapeutically effective dose of polymer conjugates containing biologically active hGH.

Human Growth Hormone

The term human Growth Hormone (hGH) as used herein encompasses human Growth Hormone and also variants of hGH such as analogs, fragments, homologs, deriviatives or allelic variants of hGH which have the same function as the naturally occurring polypeptide. Growth Hormone according to the invention may be purified from human or animal sources, produced chemically or recombinantly. Preparations of hGH are commercially available. Recombinantly produced hGH may be referred to as Syntropin.

The manufacture of recombinant hGH species is well known and is taught by U.S. Pat. Nos. 6,566,328; 5,962,411 & 5,334,531 which are incorporated herein by reference. hGH to which at least one PEG has been attached may be referred to herein as “pegylated hGH”, PEG-hGH, pegylated syntropin or PEG-syntropin.

Preparation of Biocompatible Polymer-Biologically Active Material Conjugates

To conjugate biocompatible polymers to a biologically active molecule such as hGH, one of the end groups of polymers is converted into a reactive functional group. This process is referred to as “activation” and the product is called an “activated” polymer. For instance, to conjugate poly(alkylene oxides, PAO) to peptides or proteins, one of the hydroxyl end groups of the polymer can be converted into a reactive functional group such as carbonate and activated PAO is produced, which is soluble at room temperature. This group includes mono substituted poly(alkylene oxide) derivatives such as mPEG or other suitable alkyl-substitute PAO derivatives containing C1-4 end group.

The term “reactive functional group” used in the art and herein is the group or the residue activating biocompatible polymers to bind with biologically active materials, such as hGH.

The reactive functional group of the present invention is selected from the functional groups able to react with carboxylic acid and reactive carbonyl group, for example, primary amine, or hydrazine and hydrazide functional groups (such as acyl hydrazide, carbazate, semicarbazate, thiocarbazate etc.).

The term “coupling reagent of carboxyl group” (hereinafter referred to as coupling reagent) used in the art and herein means any reagent to couple the carboxyl groups of biologically active materials such as hGH to biocompatible polymers which have been activated at the above reactive functional group.

The coupling reagents of the carboxyl group in the present invention of interest include, but are not limited to, carbodiimidyl coupling agents, for example, EDAC[N-(3-dimethyl-aminopropyl)-N′-ethylcarbodiimide hydrochloride], DIC[1,3-diisopropyl carbodiimide], DCC[dicyclohexyl carbodiimide], and EDC[1-ethyl-3-(3-dimethylamino propyl)-carbodiimide]. The preferable coupling agent for the carboxyl group is EDAC.

The method of preparing the conjugates of the present invention includes the step of reacting biologically active molecules containing nucleophiles capable of performing the substitution reaction with activated biocompatible polymers under conditions in which sufficient conjugation can be possible while retaining at least a portion of intrinsic bioactivity of biologically active molecules.

The biologically active material-biocompatible polymer conjugates with a ratio of 1:1 are obtained by reacting the biologically active materials with a stoichiometric excess amount of polymers. For example, in the preparation of protein-polymer, peptide-polymer, enzyme-polymer, antibody-polymer, and drug-polymer conjugates, the molar ratio of biologically active material to biocompatible polymer is in the range of from about 1:1 to 1:20, more preferably from 1:1 to 1:10. The reagents to activate carboxyl groups of biologically active materials are selected from the group as follows, but are not limited to them. For example, N-(3-dimethyl-aminopropyl)-N′-ethylcarbodiimide hydrochloride (EDAC), water soluble carbodiimide group such as 3-[2-morpholinyl-(4)-ethyl], and 5-substituted isoxazolinium salts such as p-toluene sulfonate, Woodward's Reagent K.

The molar ratio of biologically active materials to EDAC used in the present invention is in the range of from about 1:1 to 1:50, more preferably from about 1:1 to 1:30, and most preferably from about 1:1 to 1:20. However, increased yield of PEG-biologically active material conjugates was observed when the addition of EDAC was divided to more than 5 times, preferably 5 or 6 times rather than adding 20-fold molar excess of EDAC at once because EDAC is readily hydrolyzed in aqueous solution.

The conjugation reaction of biologically active materials such as hGH with activated polymers is dependent on the pH of water soluble solvents functioning as a buffer. In general, the pH of reaction buffer for proteins/polypeptides is in the range from 2 to 5, preferably from 2.5 to 4.5. The optimum reaction condition for stabilization of these substances and reaction yield has been known in the art. The suitable temperature for the conjugation reaction is in the range of 0 to 60° C. and preferably in the range of 4 to 30° C. The temperature of the solvents should not exceed the denaturation temperature of proteins or peptides. Also, the reaction time of 10 minutes to 5 hours is preferable in this preparation. The conjugates prepared can be recovered and purified by column chromatography, diafiltration or a combination of above two processes.

Pharmaceutical Composition

The present invention also relates to a pharmaceutical composition comprising a therapeutically effective dose of the activated biocompatible polymer-biologically active material conjugates as an active ingredient.

The term “pharmaceutically acceptable” used in the art and herein means not causing allergic reaction or similar reaction when administered to humans.

The biocompatible polymer-biologically active hGH conjugate as an active ingredient of the pharmaceutical composition can be used itself or formulated in combination with pharmaceutically acceptable carriers for disease prevention and treatment.

The term “pharmaceutically acceptable carrier” used in the art and herein means pharmaceutically acceptable molecules, composition, or vehicles such as solutions, diluents, excipients, or solvents to carry the biologically active materials such as hGH from one organ or tissues to other organs or tissues. The pharmaceutical composition of the present invention can be administered by the route of oral, local, injection or parenteral route and its formulation include therapeutically effective doses of the biocompatible polymer-biologically active material conjugates as an active ingredient. The formulation for oral administration of the present invention include pills, tablets, coated tablets, granules, troches, wafers, elixirs, hard and soft gelatin capsules, solutions, syrups, emulsions, suspensions, or sprays etc. and for parenteral administration, injectable solutions, microcapsules, patches, and others are included.

The pharmaceutical formulation can be prepared according to the known method by using pharmaceutically acceptable inactive inorganic or organic additives. For example, lactose, corn starch and its derivatives, talc, or stearic acid and its salts can be used to prepare pills, tablets, and hard gelatin capsules. The additives of soft gelatin capsules and suppositories are for example, oil, wax, semi-solid or liquid polyol, and natural or solidified oil. The suitable additives for preparation of solution or syrup are for example, water, sucrose, invertase, glucose, and polyol. The suitable additives for preparation of injectable solution are water, alcohol, glycerol, polyol, plant oil etc. The injectable solution can be used as the combination of preservatives, indolent agents, solubilizers, and stabilizers. The formulation for local administration can be also used as the combination of gas, diluents, lubricants, and preservatives. The suitable additives for microcapsules or transplantation are copolymer or glycolic acid and lactic acid.

The dose of the biocompatible polymer-biologically active material conjugates of the present invention varies depending on the absorption rate of the biologically active materials, solubility, patient's age, sex, condition and severity of diseases, etc. as well known in the art. In Example 26 shown below, pegylated hGH proteins (hGH) that retain up to 20% of the activity of native hGH and have significantly larger (10-fold higher) half-lives in the circulation of animals are shown. In preferred embodiments, PEG-hGH retains at least 1%, more preferably 5%, yet more preferably 10% and yet more preferably 15% of the native activity. Preferred embodiments retain 10-20% of the activity of the native hGH.

In preferred embodiments, pegylated hGH according to the invention has at least 3 fold, preferably at least 5 fold, yet more preferably at least 7 fold and yet more preferably at least 10 fold greater half lives in circulation in vivo compared to native hGH.

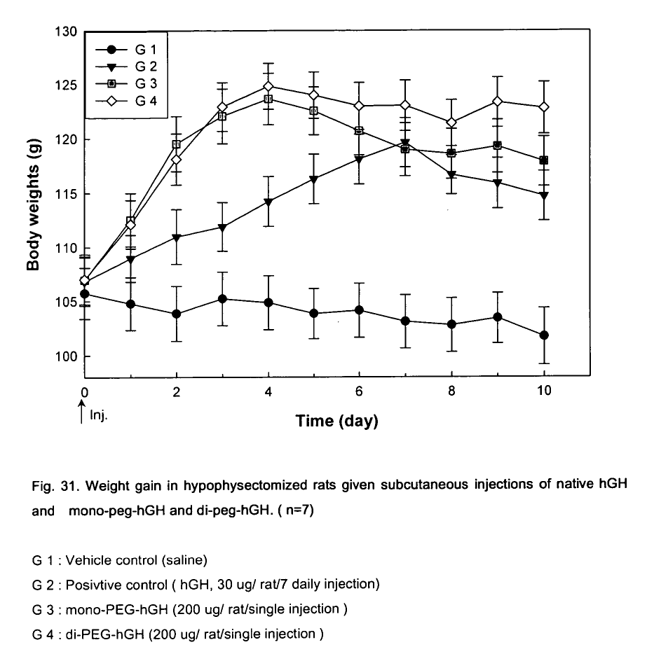

PEG-hGH clearly has the potential to show clinical utility combined with a much easier form of administration. In preferred embodiments, administration of pegylated hGH is less than daily, preferably no more than 5 times per week, more preferably no more than 4 times per week, yet more preferably no more than 3 times per week, yet more preferably no more than 2 times per week, and yet more preferable no more than one time per week. As shown in an animal weight gain assay in FIG. 31, a weekly injection of pegylated hGH or Syntropin (recombinant hGH) is equivalent in potency to daily injections of native hGH, which is the component of all brands currently in the marketplace. According to the Human Growth Foundation, it is estimated that 10,000-15,000 children in the United States have growth failure due to growth hormone deficiency. Clearly this demonstrates a need for a slow release form of hGH.

Particularly, the administration of biocompatible polymer-biologically active material conjugates of the present invention reduces the injection intervals from daily or once per two days to weekly or biweekly injection. Therefore, the toxicity and site effects of drugs by frequent administration are reduced substantially.

Therapeutic Uses

Any condition amenable to treatment by unmodified growth hormone (GH) may be treated with PEG-hGH according to embodiments of the invention. In particular, PEG-hGH according to embodiments of the invention may be used to treat children with growth hormone (GH) deficiency, generally defined as a growth in height of less than 2 inches per year, although more extensive testing confirms a growth hormone deficiency. This GH deficiency may be due to a congenital problem, a tumor, infection or radiation treatment such as for tumors to the head and neck.

PEG-hGH according to embodiments of the invention may also be used for treatment of the results of interuterine growth restriction. In some cases, an infant may be small for its gestation time due to maternal nutrition, infectious disease, environment, excess maternal alcohol consumption or other factors. Administration of PEG-hGH allows children suffering from this disorder to catch up to their peers in growth.

PEG-hGH according to embodiments of the invention may be used in treatment of chronic renal insufficiency in children as hGH is effective in stimulating growth.

PEG-hGH according to embodiments of the invention may be used to treat Turner syndrome. Although Turner syndrome is not caused by GH deficiency, administration of GH may allow girls afflicted with Turner syndrome to reach a normal height. PEG-hGH according to embodiments of the invention may be used in treatment of symptoms of Prader-Willi syndrome to increase growth and lean body mass and decrease body fat.

PEG-hGH according to embodiments of the invention may be used to treat idiopathic short stature, that is, height that is well below average for a child's age and sex. For children who do not present with a growth hormone deficiency and are normal physically, but more than two standard deviations below normal height, PEG-hGH according to embodiments of the invention may be used to increase height in these children.

Children who have growth hormone therapy as children often benefit from this therapy as adults as well. As adults they may not need to grow taller, but may still be deficient in growth hormone which leads to excess fat, decreased muscle mass and low vitality. In addition, some adults who did not have growth hormone therapy as children may produce insufficient amounts of growth hormone as adults. Symptoms of a GH deficiency include increased fat around face and abdomen, low level of lean body mass, bone loss, thinning skin with fine wrinkles, poor sweating or body temperature regulation, low interest in sex, sleep problems, poor muscle strength, poor exercise performance, high cholesterol levels, production of too much insulin and depression. PEG-hGH according to embodiments of the invention may be used in treatment programs for these patients.

GH has been FDA approved for use in adults for treatment of wasting syndrome due to AIDS, burns or traumatic injuries. PEG-hGH according to embodiments of the invention may be used to treat these conditions.

GH has also been shown to be useful to combat effects of aging. As part of the aging process, the production of GH diminishes. GH depletion is marked by the usual signs of aging, which includes increased body fat (especially around the waist), reduced vitality, decreased muscle mass, increased blood pressure and cholesterol and poor general health. PEG-hGH according to embodiments of the invention may be used to treat these symptoms.

Conjugated hGH as described above may be conveniently provided to the patient or health care practitioner as a kit. The kit includes the hGH conjugate, preferably in a pre-measured dose form. The kit preferably includes one or more containers of hGH conjugate as a pre-measured dose.

The hGH conjugate would be provided in lyophilized form or in a pharmaceutically acceptable carrier. If the hGH conjugate is provided in lyophilized form, the kit would preferably also include a pharmaceutically acceptable carrier for reconstitution of the conjugate.

In preferred embodiments, the kit would include a delivery device for delivering the hGH to the individual being treated. The delivery device is preferably a syringe. In some embodiments, the kit may include a syringe preloaded with the pre-measured dose of hGH conjugate.

In some preferred embodiments, the kit may also include any of a skin antiseptic for treatment of skin before delivery using the delivery device or syringe, bandaging material and instruction sheet.

The following examples further describe and demonstrate embodiments within the scope of the present invention. The examples are given solely for the purpose of illustration and are not to intended limit the present invention, as many variations thereof are possible without departing from the spirit and scope of the invention.

### EXAMPLES

1. Preparation of Biocompatible Polymer-Biologically Active Material Conjugates via a Carboxyl Group of Biologically Active Material

### Example 1

### Preparation of mPEG (12000)-Hz-G-CSF Conjugate

1 mg of G-CSF solution (0.00005 mmol, Dong-A Pharm. LEUCOSTIM) was dialyzed (Centricon-10, Amicon, USA) against 50 mM MES buffer solution (pH 3.0) to the final concentration of 2 mg/ml. To this protein solution, 6.6 mg of mPEG(12000)-hydrazide (Hz) (ISU Chemical, Korea, 0.0005 mmol) was added and followed by 2 ul (0.001 mmol, 20-fold molar excess) of EDAC solution prepared by dissolving 2 mg of EDAC in 20 ul of d-H2O. The reaction was carried out for 1 hour at room temperature (20-25° C.) with stirring. After 1 hour, unreacted G-CSF and excess reagent were removed by size exclusion column or ion-exchange column. More than 0.3 mg of mPEG(12000)-Hz-G-CSF conjugate was obtained. By changing the amount of EDAC from 20 to 200-fold molar excess and mPEG(12000)-Hz from 10 to 20-fold molar excess, the reaction was repeated. It was observed that two or more mPEG(12000)-Hz were attached to the carboxyl group of G-CSF when the amount of EDAC is over 50-fold molar excess.

### Example 2

### Preparation of mPEG(5000)-Hz-G-CSF Conjugate

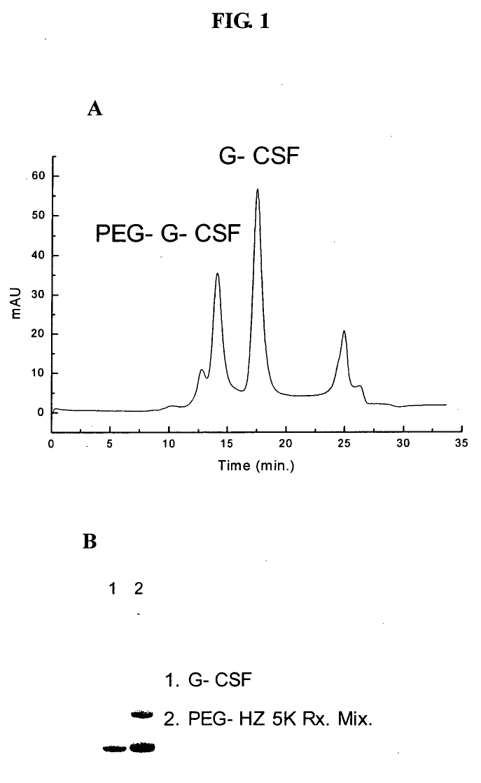

1 mg of G-CSF solution (0.00005 mmol) was dialyzed (Centricon-10, Amicon, USA) against 50 mM MES buffer solution (pH 3.0) to the final concentration of 5 mg/ml. To this protein solution, 1.3 mg of mPEG(5000)-Hz (ISU Chemical, Korea, 0.00025 mmol) was added, followed by 2 ul (0.001 mmol, 20-fold molar excess) of EDAC solution prepared by dissolving 2 mg of EDAC in 20 ul of d-H2O. The reaction was carried out for 1 hour at room temperature (20-25° C.) with stirring. After 1 hour, unreacted G-CSF and excess reagent were removed by size exclusion column or ion-exchange column. More than 0.3 mg of mPEG(5000)-Hz-G-CSF was obtained. FIG. 1 shows the production of mPEG(5000)-Hz-G-CSF conjugate by SDS-PAGE and HPLC profile (size exclusion chromatography).

### Example 3

### Preparation of mPEG(20000)-Hz-G-CSF Conjugate

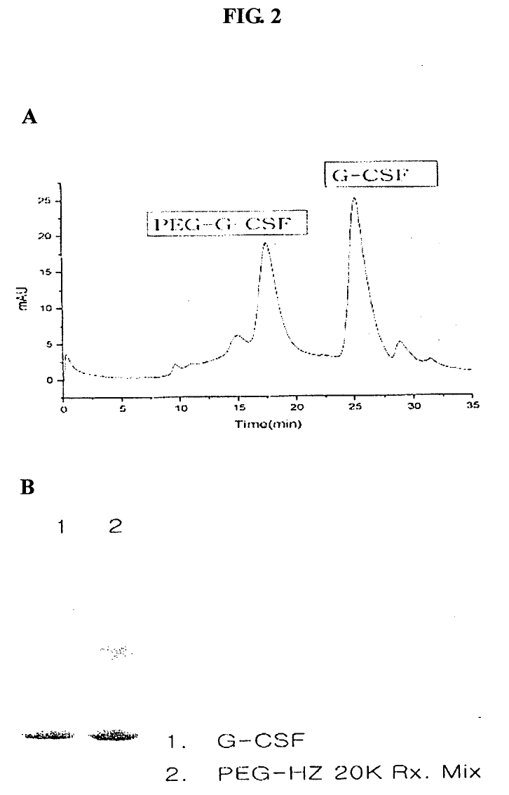

1 mg of G-CSF solution (0.00005 mmol) was dialyzed against 50 mM MES buffer solution (pH 3.0) by ultrafiltration (Centricon-10, Amicon, USA) to the final concentration of 5 mg/ml. To this protein solution, 5 mg of mPEG(20000)-Hz (ISU Chemical, Korea, 0.00025 mmol) was added, followed by 2 ul (0.001 mmol, 20-fold molar excess) of EDAC solution prepared by dissolving 2 mg of EDAC in 20 ul of d-H2O. The reaction was carried out for 1 hour at room temperature (20-25° C.) with stirring. After 1 hour, unreacted G-CSF and excess reagent were removed by size exclusion column or ion-exchange column. More than 0.3 mg of mPEG(20000)-Hz-G-CSF, was obtained. FIG. 2 shows the production of mPEG(20000)-Hz-G-CSF conjugate by SDS-PAGE.

### Example 4

### Preparation of mPEG(5000)-Hz-IFN Conjugate

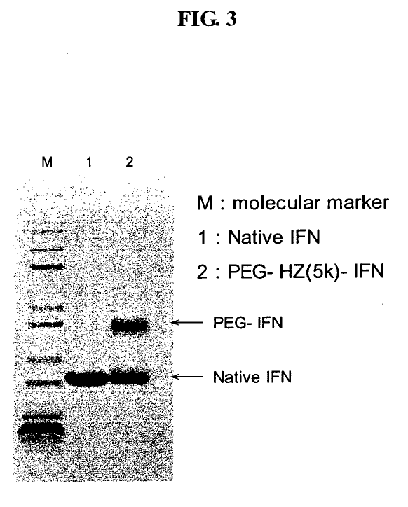

Four tubes each containing 200 ug of IFN solution (0.00001 mmol, Korea Green Cross Corp., Green Alpha) were dialyzed (Centricon-10, Amicon, USA) respectively, against 50 mM MES buffer solution (pH 3.0) to the final concentration of 1 mg/ml. To each tube, 2.16 mg of mPEG(5000)-HZ was added, followed by 0.8 ul (40-fold molar excess) of EDAC solution prepared by dissolving 2 mg of EDAC in 20 ul of d-H₂O. The reaction was carried out for 1 hour at room temperature (20-25° C.) with stirring. After 1 hour, unreacted IFN and excess reagent were removed by size exclusion column or ion-exchange column. FIG. 3 shows the conjugation of mPEG(5000)-HZ to IFN molecules by SDS-PAGE.

### Example 5

### Preparation of mPEG(12000)-Hz-IFN Conjugate

1 mg of IFN solution (0.00005 mmol) was dialyzed (Centricon-10, Amicon, USA) against 50 mM MES buffer solution (pH 3.0) to the final concentration of 1 mg/ml. To this protein solution, 6.6 mg of mPEG(12000)-Hz (0.0005 mmol) was added, followed by 2 μl (0.001 mmol, 20-fold molar excess) of EDAC solution prepared by dissolving 2 mg of EDAC in 20 ul of d-H₂O. The reaction was carried out for 1 hour at room temperature (20-25° C.) with stirring. After 1 hour, unreacted IFN and excess reagent were removed by size exclusion column or ion-exchange column. More than 0.3 mg of mPEG(12000)-Hz-IFN was obtained.

### Example 6

### Preparation of mPEG(20000)-Hz-IFN Conjugate

Four tubes containing 200 ug of IFN solution (0.00001 mmol) in each tube were dialyzed (Centricon-10, Amicon, USA) against 50 mM MES buffer solution (pH 3.0) to the final concentration of 2 mg/ml. To each tube, 4.32 mg of mPEG(20000)-Hz (0.0002 mmol, ISU Chemical, Korea) was added, followed by 1 ul (50-fold molar excess) or 4 ul (200-fold molar excess) of EDAC solution prepared by dissolving 2 mg of EDAC in 20 ul of d-H₂O. In addition, 30-fold molar excess of sulfo-NHS was added to accelerate the reaction and the results were compared. The reaction was carried out for 1 hour at room temperature (20-25° C.) with stirring. The reaction condition of each sample is described in the table below. After 1 hour, unreacted IFN and excess reagent were removed by size exclusion column or ion-exchange column. The yield of each reaction was compared by SDS-PAGE.

TABLE 1

| Sample | | | | Rx. |
|---|---|---|---|---|
| No. | IFN | mPEG-Hz | Buffer | Time |
| #1 | 2 mg/ml | 20K(×20) | 50 mM MES pH 4.4 | 1 hr |
| | | | EDAC(×50) | |
| #2 | 2 mg/ml | 20K(×20) | 50 mM MES pH 4.4 | 1 hr |
| | | | EDAC(×50) | |
| | | | NHS(×30) | |
| #3 | 2 mg/ml | 20K(×20) | 50 mM MES pH 4.4 | 1 hr |
| | | | EDAC(×200) | |

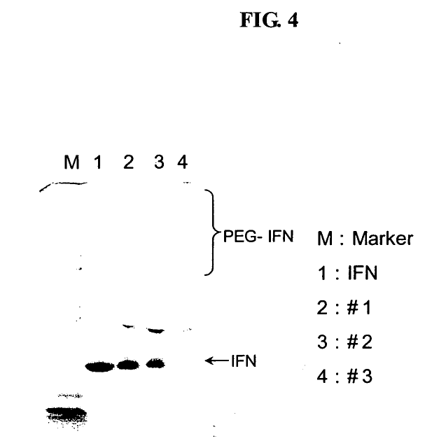

As a result, the reaction with 200-fold molar excess of EDAC showed that too many PEGs were attached to the carboxyl group of IFN and separation of the PEG-IFN conjugate with the molar ratio of 1:1 was not successful and estimation of the number of PEGs attached was not easy. Also, the reaction with 50-fold molar excess of EDAC proceeded but PEG-IFN conjugate with the ratio of 1:1 was not readily distinguishable because the PEG-IFN conjugate was diffused on SDS-PAGE due to the excess amount of EDAC used. When sulfo-NHS was added to the reaction to enhance the efficiency, there was no difference from a control in which sulfo-NHS was not added (FIG. 4).

Also, the reaction efficiency upon adding EDAC several times was performed according to reaction conditions as described in Table 2 below.

TABLE 2

| Sample | | | | Rx. |
|---|---|---|---|---|
| No. | IFN | mPEG-Hz | I. BUFFER | Time |
| #1 | 5 mg/ml | 20K(×5) | 50 mM MES pH 4.4 | 1 hr |
| | | | EDAC(×10) added | |
| | | | 6 times every 10 min. | |
| | | | Total molar excess | |
| | | | of EDAC: 60 times | |
| #2 | 5 mg/ml | 20K(×5) | 50 mM MES pH 4.4 | 1 hr |
| | | | EDAC(×10) added | |
| | | | 5 times every 10 min. | |
| | | | Total molar excess | |
| | | | of EDAC: 50 times | |
| #3 | 5 mg/ml | 20K(×5) | 50 mM MES pH 4.4 | 1 hr |
| | | | EDAC(×5) added | |
| | | | 6 times every 10 min. | |
| | | | Total molar excess | |
| | | | of EDAC: 30 times | |
| #4 | 5 mg/ml | 20K(×5) | 50 mM MES pH 4.4 | 1 hr |
| | | | EDAC(×3) added | |
| | | | 6 times every 5 min. | |
| | | | Total molar excess | |
| | | | of EDAC: 18 times | |

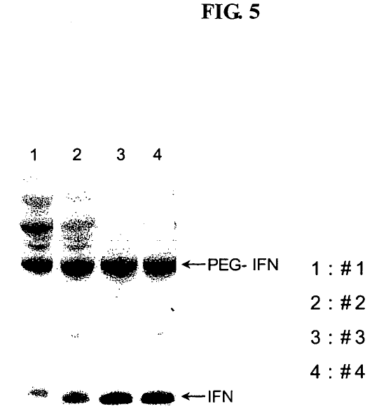

As a result, when EDAC was added stepwise to the reaction mixture, the yield of mPEG-Hz-IFN conjugate with the molar ratio of 1:1 was high (FIG. 5). When the amount of EDAC was over 50-fold molar excess although addition of EDAC was performed stepwise, two or more PEGs were randomly attached to the IFN as shown in FIG. 5.

### Example 7

### Preparation of mPEG(20000)-Hz-IFN Conjugate

1 mg of IFN solution (0.00005 mmol) was dialyzed (Centricon-10 (Amicon, USA)) against 50 mM MES buffer solution (pH 2.5) to the final concentration of 5 mg/ml. To this protein solution, 10.8 mg of mPEG(20000)-Hz (0.0005 mmol, 10-fold molar excess) was added, followed by 2 μl (0.001 mmol, 20-fold molar excess) of EDAC solution prepared by dissolving 2 mg of EDAC in 20 ul of d-H₂O. The reaction was carried out for 1 hour at room temperature (20-25° C.) with stirring. After 1 hour, unreacted IFN and excess reagent were removed by size exclusion column or ion-exchange column. More than 0.3 mg of mPEG (20000)-Hz-IFN conjugate was obtained.

### Example 8

### Purification of mPEG(20000)-Hz-IFN Conjugates With the Molar Ratio of 1:1

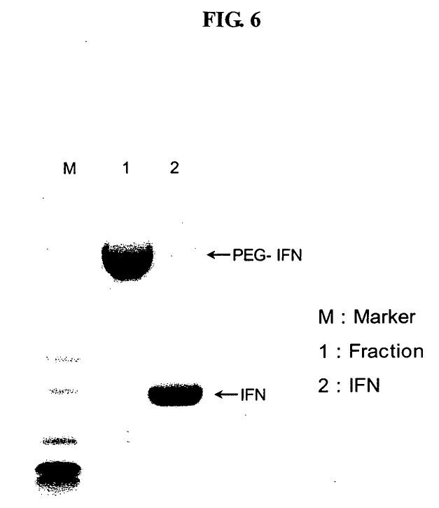

mPEG(20000)-Hz-IFN conjugate (Example 6) was diluted with 10 mM sodium acetate buffer (pH4.4) to the final concentration of 1 mg/ml. mPEG(20000)-Hz-IFN reaction mixture was loaded onto SP-sepharose Fast Flow column (5×50 mm, total 1 ml column vol.), which had been previously equilibrated with 10 mM sodium acetate buffer solution (pH 4.4). After washing the column with 3 column volumes of 10 mM acetate buffer (pH 4.4), a gradient of 10 mM acetate buffer (pH 4.4) containing 500 mM NaCl was used to separate mPEG(20000)-Hz-IFN with the molar ratio of 1:1 from unreacted intact IFN. The above purified mPEG(20000)-Hz-IFN was confirmed to be the conjugate wherein one PEG was attached to the carboxyl group of IFN (FIG. 6).

### Example 9

### Determination of Biological Activity of PEG-G-CSF Conjugate CPE

Cytopathic effect assay was performed as follows. 2.5×10⁶ cells (5×10⁵ cells/ml) of M-NFS-60 were sub-cultured on 60 mm dishes (RPMI-1640 media, 10% FBS, 37° C., 5% CO₂). Each of native G-CSF (control) and mPEG(20000)-Hz-G-CSF (Example 3) was diluted to the concentration of 1 ng/μl and added to the 96 well plate containing 1×10⁴ cells in each well, followed by serial dilution. The plate was incubated at 37° C. for 2 days. Then each well was treated with 50 μl of XTT kit (Roche, Germany) and incubated for another 4 hours at 37° C., and O.D. value of the plate was read at 490 nm using ELISA reader.

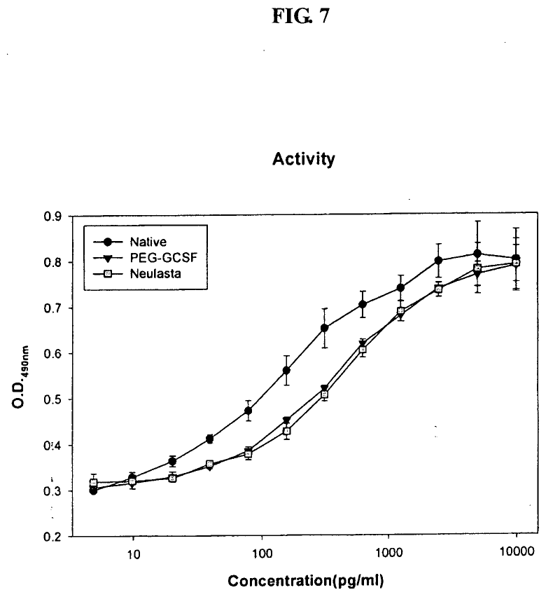

As a result, mPEG(20000)-Hz-G-CSF of the present invention was shown to retain a similar activity to mPEG(20000)-G-CSF conjugate, wherein PEG was attached to the amino group of G-CSF (FIG. 7).

### Example 10

### Determination of Half Life of PEG-G-CSF Conjugate

7-week old Sprague-Dawley rats (5 rats per group) weighing 220-240 g were anesthetized using Ketamin/Rompun and a PE tube was inserted to the vena cava of each rat by surgery. After the rat recovered, 100 ug/kg of mPEG(20000)-Hz-G-CSF (Example 3) was administered through intravenous injection. PBS and 100 ug/kg of native G-CSF were used as placebo and control, respectively, for comparison.

300 ul of blood was withdrawn through the cannula at time intervals of 0, 0.5, 1, 2, 4, 6, 12, 24, and 48 hours after injection. The serum was separated by centrifugation (13,000 rpm, 10 min, 4° C.) and stored at −20° C. for further study.

After incubation the cells for 24 hours with G-CSF free media, each well of 96 well plate was added with 1.5×10⁴ cells. Each serum sample stored as described above was diluted 100 times and 50 ul of the diluted samples was added to each well of 96 well plate. The dishes were incubated at 37° C. for 48 hours under CO₂ gas. Then each well was treated with 50 μl of XTT kit (Roche, Germany). Plates were incubated for another 4 hours at 37° C. and the O.D. value of the plate was read at 490 nm using an ELISA reader.

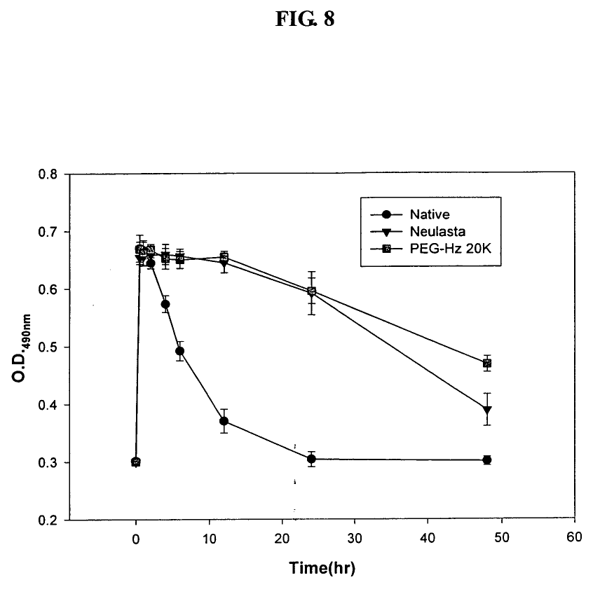

The half-life of mPEG(20000)-Hz-G-CSF (Example 3) is compared to that of native G-CSF and Neulasta™ (PEG attached to N-terminal of G-CSF, Amgen) in FIG. 8. mPEG(20000)-Hz-G-CSF (Example 3) of the present invention showed a much longer half-life compared to native G-CSF, and has similar half-life to that of Neulasta™.

### Example 11

### Determination of White Blood Cell (WBC) Count of PEG-G-CSF-Treated Rats

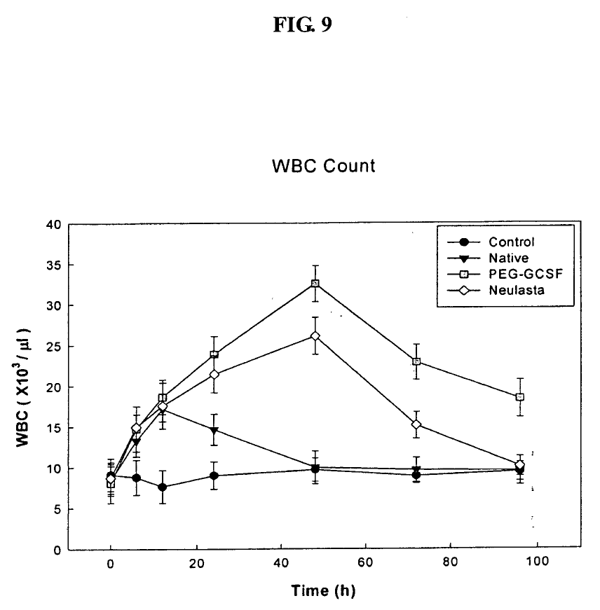

7-week old Sprague-Dawley rats weighing 220-240 g were purchased from Charles River Co. (Atsugi, Japan). 100 ug/kg of mPEG(20000)-Hz-G-CSF (Example 3) was injected to the tail vein of rats. The same amount of native G-CSF and saline solution was injected respectively as a control. Blood samples were withdrawn at time intervals of 0, 6, 12, 24, 48, 72, 96 hrs after injection through the tail vein. WBC count was measured by Automated Hematology Analyzer (Cysmex K-4500) as shown in FIG. 9. As a result, mPEG(20000)-Hz-G-CSF of the present invention showed higher WBC counts than both native G-CSF and Neulasta™.

### Example 12

### Determination of Biological Activity of PEG-IFN Conjugate

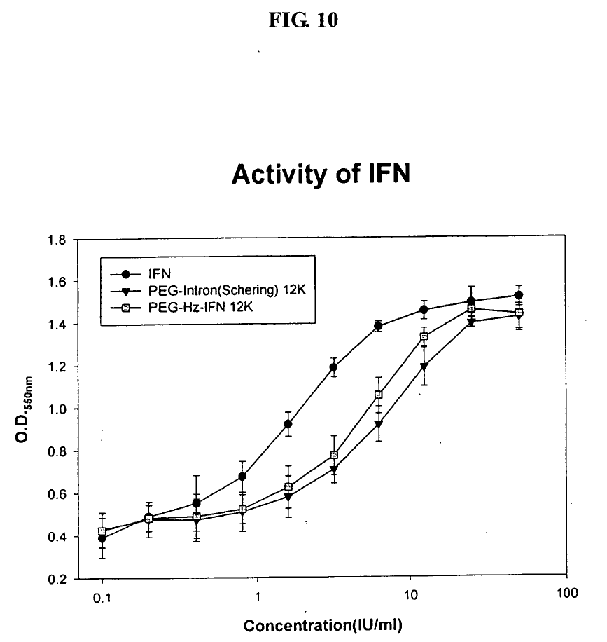

MDBK cells counted in a concentration of 7.5×10⁵ cells/ml using a hemocytometer, were suspended in 5% FBS/MEM media. mPEG(12000)-Hz-IFN (Example 5) was diluted to the concentration of 100IU (1 mg/ml=2×10⁸ IU). Each well was supplemented with 100 ul of 5% FBS/MEM media and 100 ul of the diluted samples was added to the first well followed by serial dilution. Then 100 ul of cell suspension was added to each well of 96 well plate. The dishes were incubated at 37° C. for 20 hours. 100 ul of Vesicular Stomatitis Virus (VSV, ATCC VR-158) diluted 100 times was added to each well and incubated another 20 hours at 37° C. The growth medium solution containing Vesicular Stomatitis Virus (VSV, ATCC VR-158) of 96 well plate was removed, 50 ul of 0.05% crystal violet dye solution was added to each well, and O.D. of each well was read at 550 nm by ELISA reader to determine the activity of IFN. As a result, the activity of mPEG(12000)-Hz-IFN (Example 5) was found to retain 40-50% of native IFN activity and showed activity similar to that of the comparative PEG-IFN (Schering-Plough, USA, PEG attached to amino group of IFN, approved by FDA) (FIG. 10).

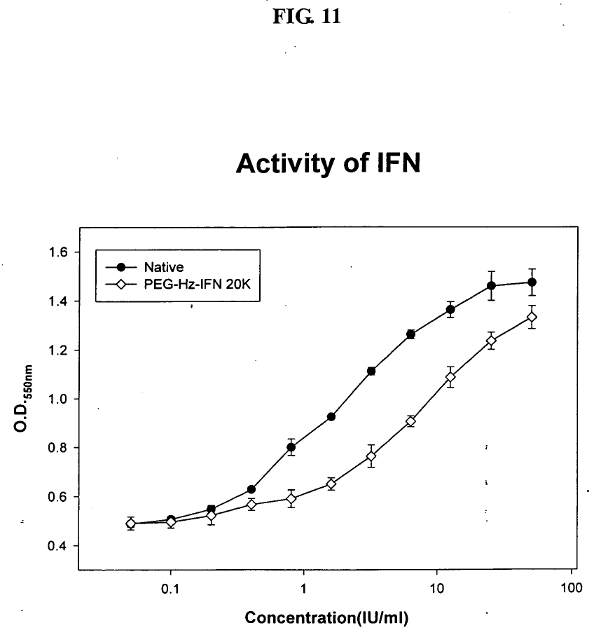

Also, the activity of mPEG(20000)-Hz-IFN (Example 6) was determined to be approximately 40% of native IFN (FIG. 11).

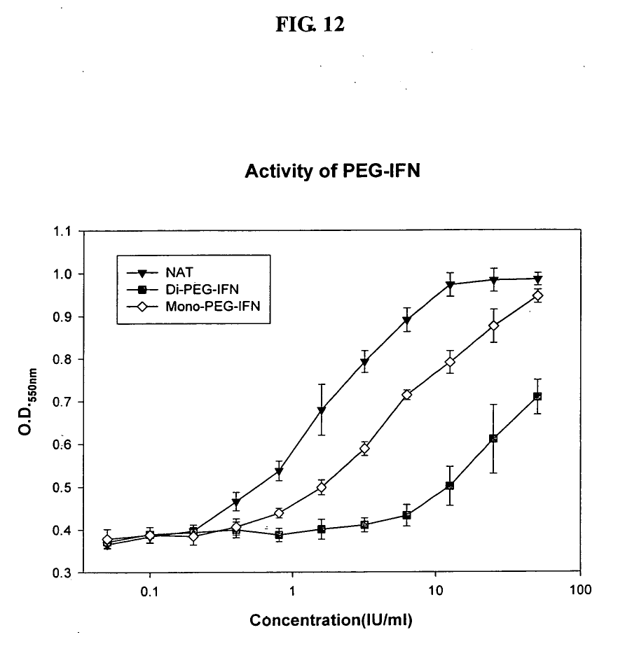

Also, the activity of Di-mPEG-Hz-IFN (two PEGs attached to one IFN) and mono-PEG-IFN(1:1 complex, one PEG attached to one IFN) was compared by CPE assay and mono-mPEG-Hz-IFN showed high biological activity (FIG. 12).

### Example 13

### Determination of Half-Life of PEG-IFN Conjugate

MDBK cells counted in a concentration of 7.5×10⁵ cells/ml were suspended in 5% FBS/MEM media. 100 ul of cell suspension was put in each well of 96 well plate. Serum was obtained after injecting mPEG(20000)-Hz-INF (Example 6) by intravenous route to rats and diluted 50 times. Each well was added with the diluted serum and incubated in a CO₂ incubator for 20 hours.

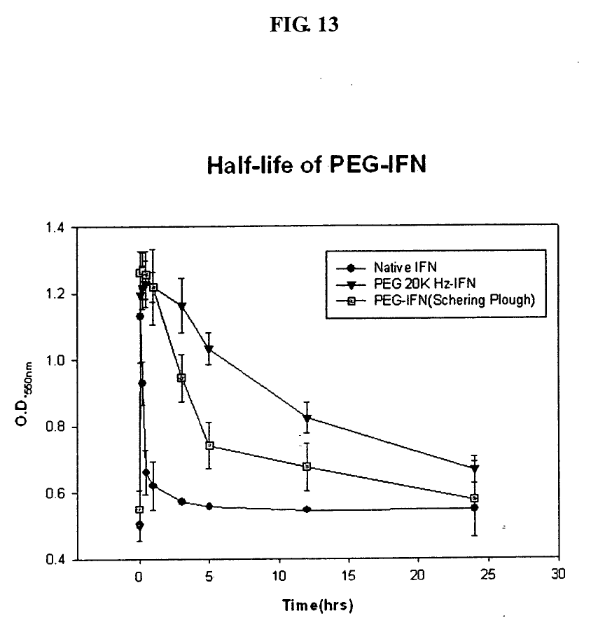

Each well was added with 100-fold diluted Vesicular Stomatitis virus (100 ul) and incubated continued for another 20 hours. The virus medium solution in each well was removed, and 50 ul of 0.05% crystal violet dye solution was added to each well. The absorbance at 550 nm was read by a Microplate reader to measure the half life of IFN. FIG. 13 shows the half-life of mPEG(20000)-Hz-IFN (Example 6) conjugated at the carboxyl group and comparison of mPEG(20000)-Hz-IFN with native IFN and comparative product, PEG-IFN. mPEG(20000)-Hz-IFN of the present invention showed a much greater half-life than native IFN and longer half-life than the comparative product, PEG-IFN.

### Example 14

### Stability of PEG-IFN Conjugate

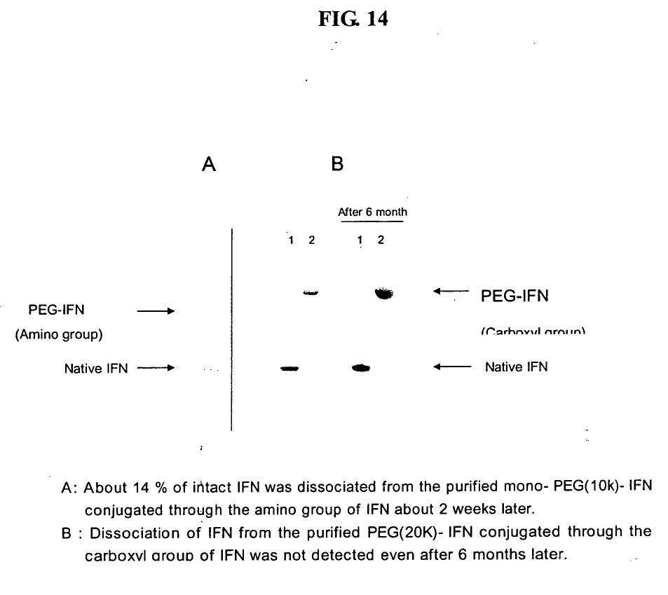

The stability of mPEG(20000)-Hz-IFN prepared and purified in Example 6 and PEG-IFN, wherein branched PEG (10K)2-NHS (attached to the amino group of IFN according to the general method in the literature followed by separating mono PEG-IFN by size exclusion column, Nektar, USA) was determined on SDS-PAGE after incubation at 4° C. in PBS solution by observing the dissociation of intact IFN on the SDS-PAGE. The concentration of each sample was adjusted to the final concentration of 1 mg/ml. As a result, it was observed that about 14% of intact IFN was dissociated from the PEG-IFN conjugated through the amino group of IFN about 2 weeks later, whereas the dissociation of IFN from PEG(20000)-Hz-IFN conjugated through the carboxyl group of IFN of the present invention was not detected even after 6 months later (FIG. 14).

### Example 15

### Preparation of mPEG(5000)-Hz-PTH Conjugate

1 mg of human PTH (parathyroid hormone, 0.00012 mmol, 1-84 aa, Dong-Kook Pharm., Korea) and 3.0 mg of activated mPEG(5000)-Hz (0.0006 mmol, ISU Chemical, Korea) in 0.5 ml of 50 mM MES buffer solution, pH 4.4 were reacted for 10 minutes with stirring at room temperature. 2.5 ul of EDAC (0.00125 mmol, 10-fold molar excess) prepared at a concentration of 100 ug/ul, was then added and reacted for 1 hour with stirring at room temperature. Unreacted mPEG(5000)-Hz and PTH were removed by using Centricon-10 (Amicon, USA) and 0.4 mg of mPEG(5000)-Hz-PTH was obtained.

### Example 16

### Preparation of mPEG(12000)-Hz-PTH Conjugates

1 mg of human PTH (0.00012 mmol) and 7.14 mg of activated mPEG(12000)-Hz (0.0006 mmol, 5 fold molar excess, ISU Chemical, Korea) in 0.5 ml of 50 mM MES buffer solution, pH 4.4 were reacted for 10 minutes with stirring at room temperature. 2.5 ul of EDAC (0.00125 mmol, 10-fold molar excess) prepared at a concentration of 100 ug/ul, was then added and reacted for 1 hour with stirring at room temperature. Unreacted mPEG(12000)-Hz and PTH were removed by using Centricon-10 (Amicon, USA) and 0.3 mg of mPEG(12000)-Hz-PTH was obtained.

### Example 17

### Preparation of mPEG(20000)-Hz-PTH Conjugates

1 mg of human PTH (0.00012 mmol) and 12 mg of activated mPEG(20000)-Hz (0.0006 mmol, 5 fold molar excess, ISU Chemical, Korea) in 0.5 ml of 50 mM MES buffer solution, pH 4.4 were reacted for 10 minutes with stirring at room temperature. 2.5 ul of EDAC (0.00125 mmol, 10-fold molar excess) prepared at a concentration of 100 ug/ul, was then added and reacted for 1 hour with stirring at room temperature. Unreacted mPEG(20000)-Hz and PTH were removed by using Centricon-30 (Amicon, USA) and 0.3 mg of mPEG(20000)-Hz-PTH was obtained.

### Example 18

### Preparation of mPEG(12000)-Hz-PTH Conjugate

1 mg of human PTH (0.00012 mmol) and 14.4 mg of activated mPEG(12000)-Hz (0.0012 mmol, 10 fold molar excess, ISU Chemical, Korea) in 0.5 ml of 50 mM MES buffer solution, pH 2.5 were reacted for 10 minutes with stirring at room temperature. 5 ul of EDAC (0.0025 mmol, 20-fold molar excess) prepared at a concentration of 100 ug/ul, was then added and reacted for 1 hour with stirring at room temperature. Unreacted mPEG(12000)-Hz and PTH were removed by using Centricon-10 (Amicon, USA) and 0.2 mg of mPEG(12000)-Hz-PTH was obtained.

### Example 19

### Preparation of mPEG(20000)-Hz-PTH Conjugate

1 mg of human PTH (0.00012 mmol) and 24 mg of activated mPEG(20000)-Hz (0.0012 mmol, 10-fold molar excess, ISU Chemical, Korea) in 0.5 ml of 50 mM MES buffer solution, pH 2.5 were reacted for 10 minutes with stirring at room temperature. 5 ul of EDAC (0.0025 mmol, 20-fold molar excess) prepared at a concentration of 100 ug/ul, was then added and reacted for 1 hour with stirring at room temperature. Unreacted mPEG(20000)-Hz and PTH were removed by using centricon-10 (Amicon, USA) and 0.2 mg of mPEG(20000)-Hz-PTH was obtained.

### Example 20

### Analysis of mPEG-Hz-PTH Conjugate

PEG-PTH conjugate and native PTH which were obtained from the above examples were characterized using the following HPLC conditions.

TABLE 3
Eluting Condition of HPLC

| | |
|---|---|
| Column: | LiChroCART 125-4 RP-8 (5 um)(Merck, USA) |
| Solvent: | A; deionized water containing 0.1% TFA, |
| | B; acetonitrile containing 0.1% TFA |
| | Flow rate: 0.8 ml/min |
| | Detector: UV detector at 220 nm |
| | Injection volume: 20 ul |

| number | Time(min) | A (%) | B (%) | Flow rate(ml/min) |
|---|---|---|---|---|
| 1 | 0.00 | 70.0 | 30.0 | 0.800 |
| 2 | 3.00 | 70.0 | 30.0 | 0.800 |
| 3 | 13.00 | 10.0 | 90.0 | 0.800 |
| 4 | 15.00 | 10.0 | 90.0 | 0.800 |
| 5 | 17.00 | 70.0 | 30.0 | 0.800 |
| 6 | 20.00 | 70.0 | 30.0 | 0.800 |

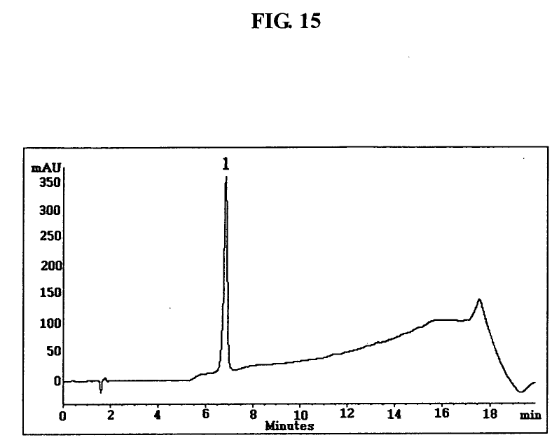

By using LiChroCART 125-4 RP-8 (5 um) for HPLC, only PTH and other proteins were detected at 220 nm whereas no PEG was detected at 220 nm. The RT of PTH was determined by HPLC. A sharp peak of PTH which is not modified by the polymer was seen around 6.8 min, then slowly increased up to about 18 min, and decreased again (FIG. 15).

mPeg-Hz-PTH prepared as described above was eluted at 6.8 min for unreacted PTH and 7.3 min for PEG-PTH, respectively, in the range of the elution conditions between number 1 and 2 in the above table.

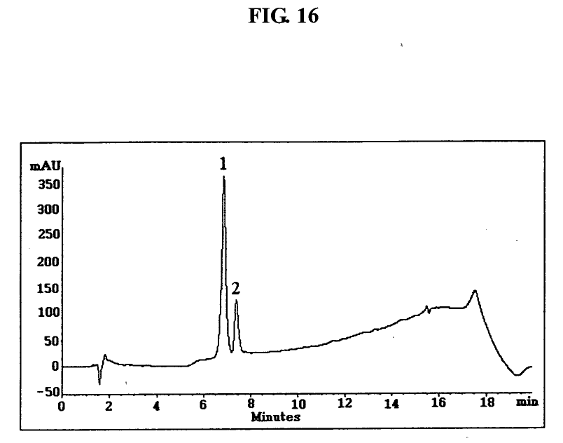

There are three kinds of products (unreacted PTH, mPEG(20000)-Hz-PTH, mPeg(2000)-Hz) present immediately after the conjugation and before purification. Only two peaks, unreacted PTH and mPEG(20000)-Hz-PTH conjugate, were detected at 220 nm on HPLC whole unreacted mPEG(20000)-Hz was not detected at 220 nm (FIG.16).

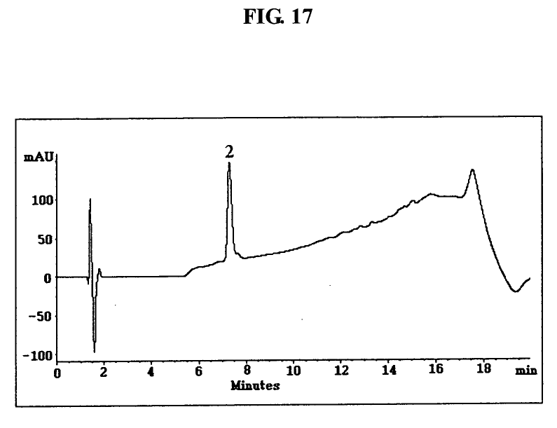

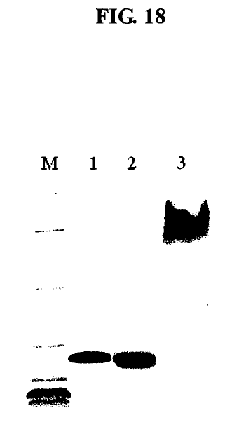

FIG. 17 shows the finally purified mPEG(20000)-Hz-PTH on HPLC and FIG. 18 SDS-PAGE stained with Coomassie blue performed after reacting mPEG(20000)-Hz with PTH.

### Example 21

### Determination of In Vitro Biological Activity of mPEG-PTH Conjugate

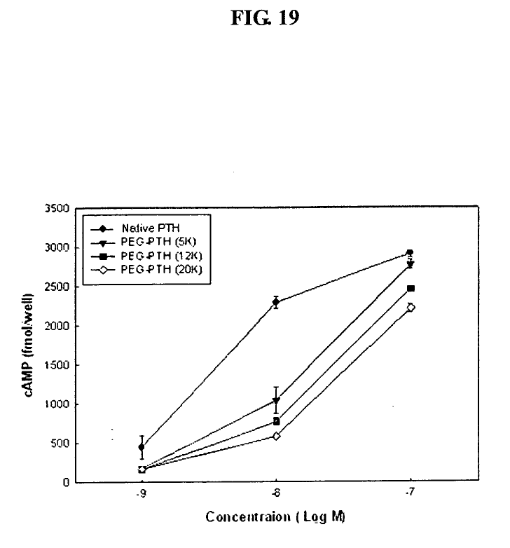

the activated mPEG-Hz having molecular weights of 5000 (5K), 12000 (12K), and 20000 (20K) were used to determine the biological activity according to molecular weight of PEG. The in vitro biological activity of native PTH, mPEG(5000)-Hz-PTH, mPEG(12000)-Hz-PTH, and mPEG(20000)-Hz-PTH was compared by determining the amount of c-AMP synthesized by c-AMP kit (Amersham Pharmacia, RPN 225, USA) using UMR-10⁶ cell line. It was found that the biological activity of mPEG-Hz-PTH was decreased as molecular weight of PEG increased. It was also found that mPEG(5000)-Hz-PTH, mPEG(12000)-Hz-PTH, and mPEG(20000)-Hz-PTH at the concentration of 10⁻⁸ mole retained 40%, 30%, and 20% activity of unreacted PTH, respectively (FIG. 19).

### Example 22

### Determination of Half-Life of mPEG-Hz-PTH

100 ug/kg of PTH and mPEG-Hz-PTH was administered through intravenous injection, respectively to each of male rats weighing 300-350 g. Blood was then withdrawn through the cannula at time intervals of 0, 5, 10, 15, 30, 60, and 120 min after injection. The serum was separated by centrifugation (10,000 rpm, 10 min, 4° C.) and the half life of mPEG-Hz-PTH was indirectly determined by calculating the concentration of remaining PTH by measuring the concentration of cAMP in plasma using c-AMP kit (Amersham Pharmacia, RPN 225, USA).

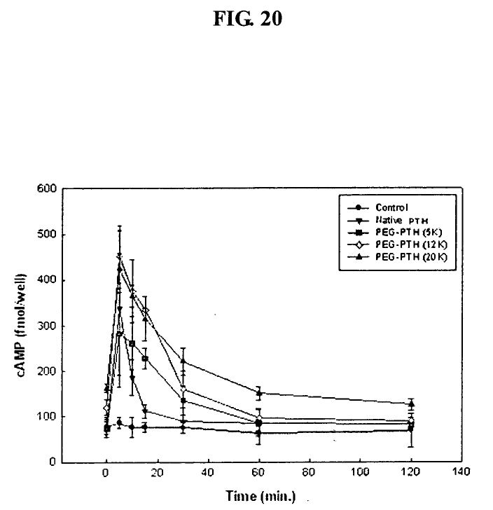

As a result, unreacted PTH and mPEG(5000)-Hz-PTH were not detected after 15 mins after administration. However, mPEG(12000)-Hz-PTH and mPEG(20000)-Hz-PTH were detected after 1 hour and 2 hours, respectively, after administration (FIG. 20).

### Example 23

### Preparation of mPEG(20000)-Hz-EPO Conjugate

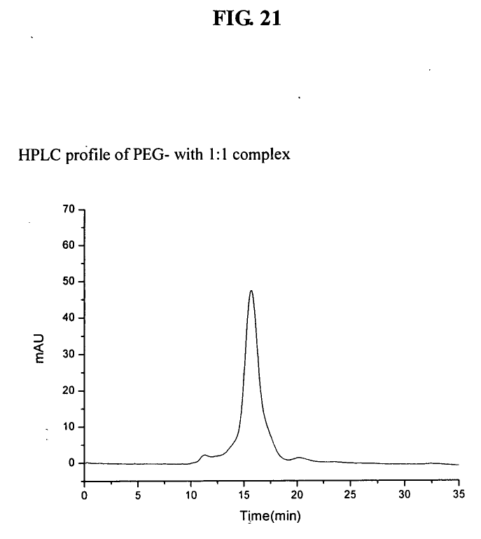

250 ug of EPO solution (0.0000083) was dialyzed (Centricon-10, Amicon, USA) against 50 mM MES buffer solution (pH 3.0) to the final concentration of 5 mg/ml. To this tube, 1.67 mg of mPEG(20000)-Hz (0.000083 mmol, ISU Chemical, Korea) was added, followed by adding 2.4 ul (30-fold molar excess) EDAC solution prepared by dissolving 1 mg of EDAC in 50 ul of d-H₂O. The reaction was carried out for 1 hour at room temperature (20-25° C.) with stirring. After 1 hour, unreacted EPO and excess reagent were removed by size exclusion column or ion-exchange column. The purified PEG-EPO with 1:1 complex was verified by HPLC in FIG. 21.

### Example 24

### Determination of Biological Activity of PEG-EPO Conjugate

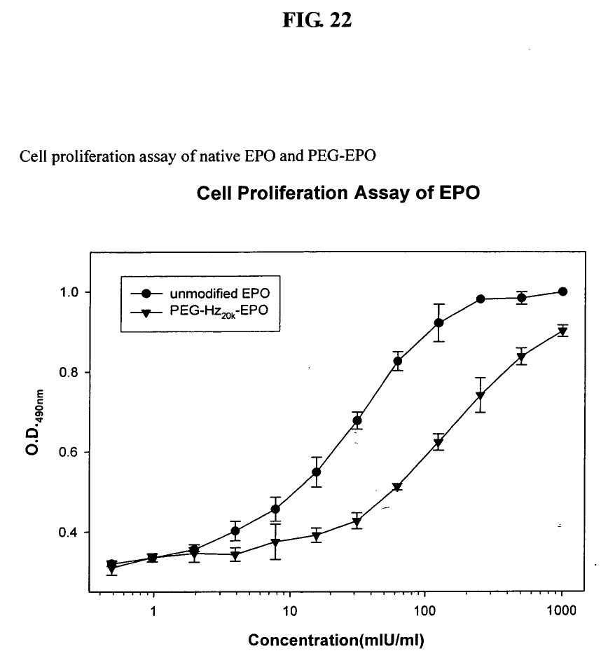

Cell proliferation assay was performed as follows. 1×10⁶ cells/ml of F-36E were sub-cultured on 60 mm dishes (RPMI-1640 media, 10% FBS, 37° C., 5% CO2). 80 ul of media (10% RPMI-1640) was added to each well. Each of native EPO (control) and mPEG(20000)-Hz-EPO (Example 23) was diluted to the concentration of 5 mu/μl and added to the 96 well plate followed by serial dilution. 20 ul of 1×10⁶ cells/ml was then added to each well followed by incubation at 37 C. for 4 days. Then each well was treated with 50 μl of XTT kit (Roche, Germany) and incubated for another 4 hours at 37° C., and O.D. value of the plate was read at 490 nm using ELISA reader (FIG. 22).

### Example 25

### Purification of Native hGH

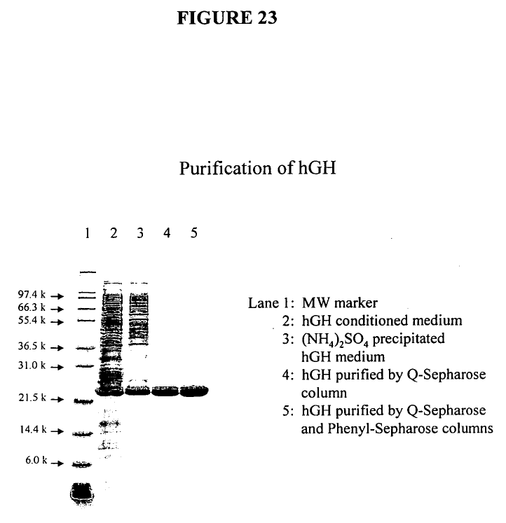

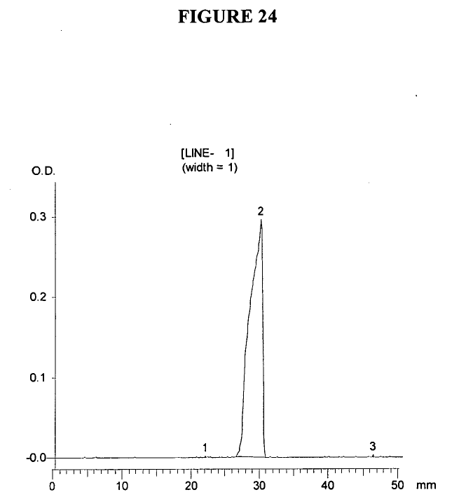

hGH was purified with ammonium sulfate precipitation, followed by purification on Q-Sepharose and Phenyl Sepharose columns. FIG. 23, displays an SDS-PAGE gel of the drug substance obtained after each successive purification step. The soluble, biologically-active growth hormone product in conditioned medium (bacterial growth media after bacterial lysis containing the released protein) is shown in lane 2 (FIG. 23). The drug substance is then subjected to the 3-step purification procedure of ammonium sulfate precipitation, followed by successive chromatography steps on Q-Sepharose and Phenyl-Sepharose. The purified, final drug substance depicted in Lane 5 of FIG. 23, was judged to be greater than 99% pure by a densitometric scan of this lane as shown in FIG. 24. This manufacturing process produces a human growth hormone product of high purity with biological activity equivalent to an international growth hormone standard. It was used in subsequent pegylation studies.

### Example 26

### Preparation of Pegylated hGH

Human Growth Hormone (PEG-hGH) (manufactured by Phage Biotech Corp.) was pegylated using the methods as described above, to characterize mono-PEG-hGH (one PEG attached to one hGH molecule, Lot No BPM#04-003) and di-PEG-hGH (two PEGs attached to one hGH molecule, Lot No BPM#04-004) by HPLC, and to evaluate PEG-hGH in vitro biological activity and half-life in rats. The mono-and di-PEG-hGH were prepared by conjugating the activated PEG derivatives to the carboxyl group of hGH produced by the method of Example 25.

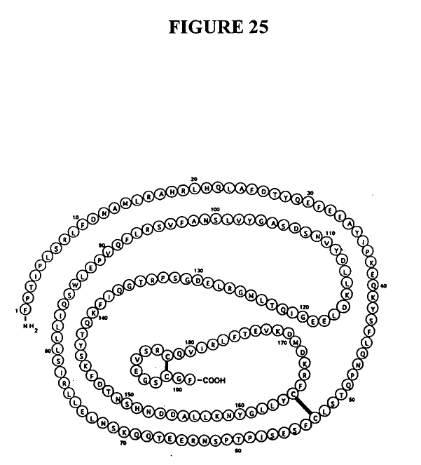

PEG-hGH was prepared by reacting hGH (FIG. 25) with activated PEG derivatives for 1 hour at 25 C. The reaction was carried out in 50 mM MES buffer at pH 3.0. EDAC was used at a 15-fold molar excess to activate PEG. The reaction was carried out using either PEG (20000) or PEG (30000). Mono-PEG-hGH and di-PEG-hGH were then purified by HPLC using a size-exclusion column. The purified PEG-hGH fractions were stored in PBS solution at 4-8 C until further analysis. PEG-hGH was verified by HPLC using a size exclusion column monitored at 220 nm. The concentration of PEG-hGH was determined by O.D. at 280 nm using UV-VIS spectrophotometer.

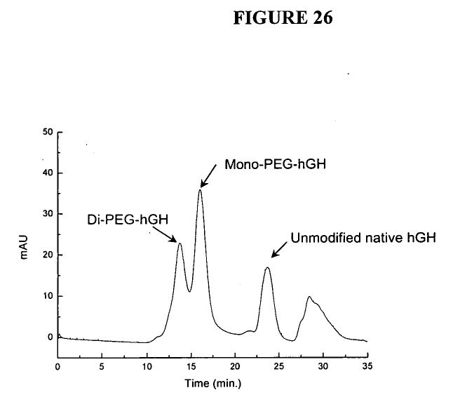

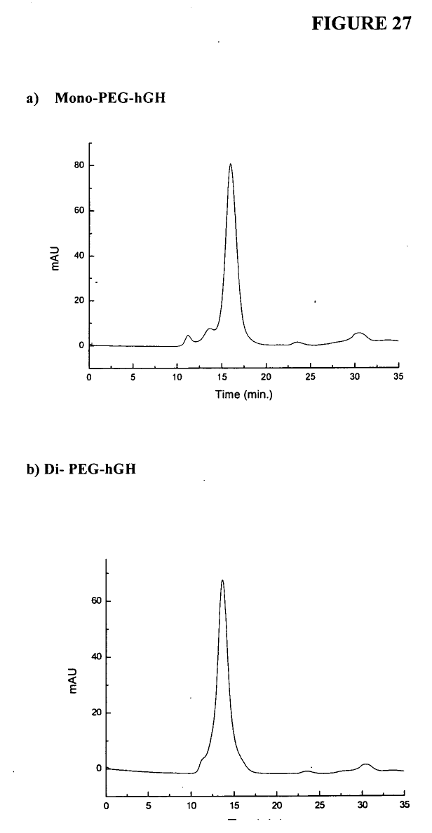

The reaction of hGH with activated PEG derivatives was verified by HPLC using a size-exclusion column as shown in FIG. 26 and approximately 48%, 31%, and 21% of mono-PEG-hGH, di-PEG-hGH, and unreacted hGH were produced, respectively. Each fraction was then purified by size-exclusion column and verified on HPLC as shown in FIG. 27A (mono-PEG-hGH) and 27B (di-PEG-hGH). The purity of each PEG-hGH samples was determined to be >95%.

Biological Activity of PEG-hGH: PK Study of PEG-hGH in Rats

The biological activity of mono- and di-PEG-hGH was determined by cell proliferation assay and compared with native hGH (Product of Phage Biotech). Mono- and di-PEG-hGH were administered to 7-week old Sprague-Dawley rats (at least 5 rats per each group) weighing 220-240 g with a dose of 200 ug/kg by s.c. injection, respectively. Native hGH was used as a control. The blood was withdrawn at a time interval of 0, 10 min, 30 min, 1 hr, 2 hr, 4 hr, 6 hr, 8 hr, 12 hr, 24 hr, 48 hr, 72 and 96 hr. post injection. The serum samples were obtained by centrifugation at 12000 rpm and stored at −20 C. for further analysis.

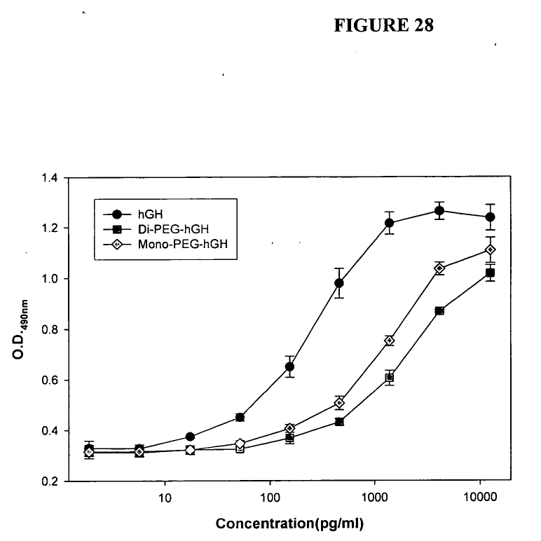

FIG. 28 shows the biological activity of the mono-and di-PEG-hGH as well as native hGH measured by cell proliferation assay. The bioassay is a cell proliferation assay, where hGH stimulates the proliferation of BaF3 cells, which have been stably transfected with the full-length human growth hormone receptor. This cell line termed Baf-B03 B2B2 has been extensively characterized (Behncken S N, et al. 1997. J Biol Chem 272:27077-27083) in terms of its response to hGH. The activity of mono- and di-PEG-hGH was determined to be 15±5% and 8±2%, respectively.

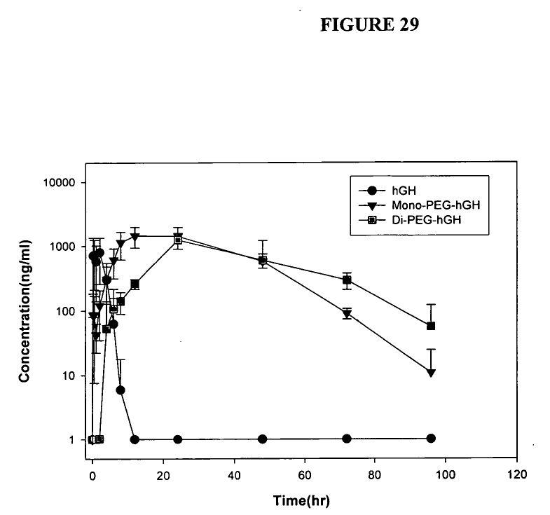

The pharmokinetic (PK) study of PEG-hGH was performed by measuring the amount of hGH in serum samples using ELISA assay compared with native hGH (Phage Biotech). FIG. 29 shows the PK study of PEG-hGH and compared to native hGH. It was shown that native hGH was cleared from the blood within 5 hours post injection whereas both mono- and di-PEG-hGH were detected after 72 hours post injection.

We observed that mono- and di-PEG-hGH retained 15±5% and 8±2% of biological activity, respectively as compared to native hGH. The PK study, however, shows that PEG-hGH was cleared much slower than native hGH in rats. Therefore, the PEG-hGH samples of this study can provide a new sustained released drug of hGH.

### Example 27

### Bioassay

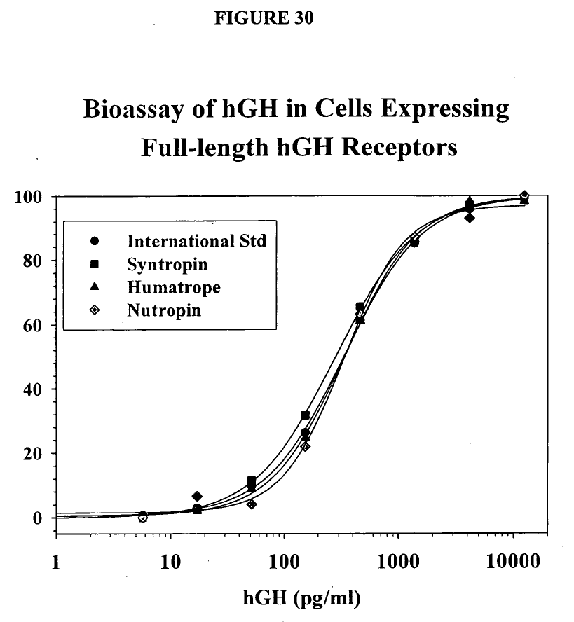

The activity of the preparation described above was tested by the cell proliferation assay with BaF3 cells and compared to commercially available hGHs. A representative standard curve is shown in FIG. 30 where similar dose response curves are seen with four commercially available hGHs. The specific activity of hGH prepared as described above compares favorably with commercially available hGH. As expected, pegylation of the native Syntropin resulted in a loss of biological activity, with a greater activity loss occurring as one attaches more peg groups to the native hGH.

TABLE 4
Potencies (IU/mg) of hGH Preparations
Tested in the Cell Proliferation Assay

| Samples | Specific Activity (IU/mg) |
|---|---|
| International Std | 3.00 |
| SYNTROPIN | 3.11 |
| Nutropin ® | 3.03 |
| Humatrope ® | 3.03 |
| Mono-peg SYNTROPIN | 0.46 |
| Di-peg SYNTROPIN | 0.25 |

A second bioassay utilized was the classical rat weight gain assay (Roswall E C, et al. 1996. Biologicals 24: 25-39) where the weight of hypophysectomized rats are monitored over a 28 day period, following subcutaneous hGH injections, once daily, for the first 7 days, or one injection at day 1 of the mono- or di-peg-SYNTROPIN. Remarkably, and unexpectedly given the rather low potency of the peg-SYNTROPINS in the cell-based bioassay (Table 4), the pegylated SYNTROPINS showed equivalent activity with native hGH when injected only once a week versus daily injections for native hGH (see FIG. 31). It can be seen in FIG. 31 that at 7 days, rats given daily injections of native hGH weighed approximately the same as rats given a single injection of either the mono-peg-SYNTROPIN or the di-peg-SYNTROPIN. Also unexpected was the rate of weight gain in the animals administered peg-SYNTROPINS versus those animals receiving native SYNTROPIN. In FIG. 31 it can be seen that the rats given a single dose of either mono-peg-hGH or di-peg-hGH gained weight at a significantly faster rate over the first 4 days than those animals receiving a daily injection of native hGH. It was not until day 7 that the animals receiving native hGH were able to “catch up” in body weight with the animals receiving the pegylated growth hormones.

Thus, while there have been described the preferred embodiments of the present invention, those skilled in the art will realize that other embodiments can be made without departing from the spirit of the invention, which includes all such further modifications and changes as come within the meaning, true scope of the claims set forth herein and equivalents thereof. The above examples further describe and demonstrate embodiments within the scope of the present invention. The examples are given solely for the purpose of illustration and are not to be construed as limitations of the present invention, as many variations thereof are possible without departing from the spirit and scope of the invention.

Application of the Present Invention

The present invention provides biocompatible polymer-biologically active material conjugates in a molar ratio of 1:1 wherein the biocompatible polymer is attached to a carboxyl group of the biologically active material such as proteins or peptides and methods of preparation thereof These conjugates have increased bioavailability and extended half-life due to their increased in vivo stability and can reduce the frequency of administration significantly when used as therapeutic drugs for diseases. In particular, pegylated hGH is provided that has up to 20% of the specific activity of the unpegylated protein and an increased half life in vivo.

1. A conjugate of biocompatible polymer-human Growth Hormone (hGH), wherein the biocompatible polymer is conjugated to a carboxyl group of hGH at a molar ratio of 1:1.

2. The conjugate according to claim 1, wherein the biocompatible polymer is selected from the group consisting of polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacrylamide, and copolymers thereof.

3. The conjugate according to claim 1, wherein the biocompatible polymer is selected from the group consisting of PEG-20000 and PEG-30000.

4. A pharmaceutical composition comprising a pharmaceutically acceptable amount of the conjugate according to claim 1 and a pharmaceutically acceptable carrier.

5. The conjugate of claim 1, wherein the carboxyl group is the C-terminus of hGH.

6. The conjugate of claim 1, wherein the activity of the conjugate is 10-20% of the activity of an unconjugated hGH protein.

7. A method of preparing a conjugate of biocompatible polymer-hGH comprising:
(a) activating a biocompatible polymer with the stepwise addition of a coupling reagent; and
(b) conjugating the activated biocompatible polymer to a carboxyl group of the hGH at a molar ratio of 1:1,
wherein the molar ratio of the hGH to the activated biocompatible polymer is 1:1 to 1:20, the ratio of the hGH to the coupling reagent is 1:1 to 1:50, and pH is in the range of 2 to 5.

8. The method according to claim 7, wherein the biocompatible polymer is activated with a reactive functional group which is able to react with a carboxylic acid and/or a reactive carbonyl group.

9. The method according to claim 7, wherein the biocompatible polymer is selected from the group consisting of polyethylene glycol, polypropylene glycol, polyoxyethylene, polytrimethylene glycol, polylactic acid and derivatives thereof, polyacrylic acid and derivatives thereof, poly(amino acid), polyurethane, polyphosphazene, poly(L-lysine), polyalkylene oxide, polysaccharide, dextran, polyvinyl pyrrolidone, polyvinyl alcohol, polyacrylamide, and copolymers thereof.

10. The method according to claim 7, wherein the biocompatible polymer is selected from the group consisting of PEG-20000 and PEG-30000.

11. The method of claim 7, wherein the carboxyl group is the C-terminus of hGH.

12. The method of claim 7, further comprising:
(c) purifying the conjugate by size exclusion chromatography;
(d) precipitating the conjugate of (c) using ammonium sulfate;
(e) purifying the conjugate of (d) by ion exchanges chromatography; and
(f) purifying the conjugate of (e) by hydrophobic interaction chromatography.

13. The conjugate of biocompatible polymer-hGH prepared according to claim 7, wherein the biocompatible polymer is conjugated to a carboxyl group of the hGH at a molar ratio of 1:1.

14. The conjugate of claim 5, wherein the biocompatible polymer is PEG.

15. The conjugate of claim 5 which has 10-20% of the activity of unconjugated hGH protein.

16. A method of treating growth failure or growth retardation by administering an effective amount of a composition comprising PEG-hGH to a patient in need thereof, wherein the PEG is conjugated to a carboxyl group of hGH at a molar ratio of 1:1.

17. The method according to claim 16, wherein the carboxyl group is the C-terminus of hGH.

18. The method of claim 16, wherein the PEG-hGH has 10-20% of the activity of unconjugated hGH protein.

19. The method according to claim 16, wherein the growth failure or growth retardation is due to homone deficiency, chronic renal disease, Turner's syndrome, cachexia or AIDS wasting.

20. The method according to claim 16, wherein the PEG-hGH is administered in combination with a pharmaceutically acceptable carrier.

21. The method according to claim 16, wherein the administration is done by injection.

22. The method according to claim 16, wherein the administration is oral.

23. The method according to claim 16, wherein the composition is administered no more than twice per week to the patient in need thereof.

24. A method of treating short stature in children by administering an effective amount of a composition comprising PEG-hGH to a patient in need thereof at a frequency of no more than twice/week, wherein the PEG is conjugated to a C-terminus carboxyl group of hGH at a molar raio of 1:1 and the PEG-hGH has 10-20% of the activity of unconjugated hGH.

25. A method of treating adverse effects associated with aging selected from the group consisting of decrease in lean muscle, increase in blood pressure, increase in cholesterol, increase in body fat, loss of skin tone, and decrease in bone density by administering an effective amount of a composition comprising PEG-hGH to a patient in need thereof, wherein the PEG is conjugated to a carboxyl group of hGH at a molar raio of 1:1.

26. A kit comprising:
the conjugate of claim 1 in lyophillized form;
a pharmaceutically acceptable carrier for reconstitution of the conjugate; and
a delivery device for delivery of the reconstituted conjugate to a patient in need thereof.

27. The kit of claim 26, further comprising:
a skin antiseptic; and
an instruction sheet.

28. A kit comprising the conjugate of claim 1 preloaded in a syringe.


SEQUENCE LISTING (1 sequence)

SEQ ID NO: 1 - protein, 192 aa, Homo sapiens
PFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF